(19) United States
(12) Patent Application Publication
(10) Pub. No.: US 2003/0064384 A1
(43) Pub. Date: Apr. 3, 2003

(54) Beta-catenin is a strong and independent prognostic factor for cancer

(76) Inventors: Mien-Chie Hung, Houston, TX (US); Weiya Xia, Missouri City, TX (US); Shiaw-Yih Lin, Houston, TX (US)

Correspondence Address: FULBRIGHT & JAWORSKI L.L.P., Melissa L. Sistrunk, Suite 5100, 1301 McKinney, Houston, TX, 77010-3095, US

(21) Appl. No.: 10/114,087

(22) Filed: Apr. 2, 2002

Related U.S. Application Data
(60) Provisional application No. 60/281,108, filed on Apr. 2, 2001.

(51) Int. Cl. C12Q 1/68; G01N 33/574

(52) U.S. Cl. 435/6; 435/7.23

(57) ABSTRACT

Cyclin D1 is one of the targets of β-catenin in breast cancer cells. Transactivation of β-catenin correlated significantly with cyclin D1 expression both in eight breast cell lines in vitro and in 123 patient samples. More importantly, high β-catenin activity significantly correlated with poor prognosis of the patients and is a strong and independent prognostic factor in breast cancer (p<0.001). Moreover, by multivariate analyses, the inventors found that activated β-catenin is a strong prognostic factor which provided additional and independent predictive information on patients survival rate even when other prognostic factors, including lymph node metastasis, tumor size, estrogen receptor and progesterone receptor status, were taken into account (p<0.001). This invention demonstrates that β-catenin is involved in breast cancer formation and/or progression and may serve as a target for breast cancer therapy.

### CROSS-REFERENCE TO RELATED APPLICATIONS

This application claims priority to United States Provisional Patent Application Serial No. 60/281,108 filed Apr. 2, 2001, incorporated by reference herein in its entirety.

### STATEMENT REGARDING FEDERALLY SPONSORED RESEARCH OR DEVELOPMENT

The government may own rights in the present invention pursuant to grant numbers R01 CA58880 and R01 CA77858 from National Cancer Institute Grant.

### BACKGROUND OF THE INVENTION

The present invention relates generally to the field of cancer prognostics. More particularly, it provides compositions and methods for use as a prognostic marker for cancer.

Cancer is a serious health issue for millions of individuals. Breast cancer is the most common form of cancer occurring in females in the US with the incidence of breast cancers in the United States projected to be 192,200 cases of invasive breast cancer diagnosed and 40,600 breast cancer-related deaths to occur in 2001 (American Cancer Society statistics). Estimates indicate that one in eight women who reach the age of 85 will develop breast cancer (American Cancer Society, Atlanta Ga., 1994, p. 13). Furthermore, ovarian cancer affects many women and includes three basic types: epithelial carcinoma, germ cell cancer, and stromal cell cancer. The most common kind of ovarian tumor is epithelial carcinoma, which starts in the epithelial cells found in the outer layer of cells in the ovaries. The second type of ovarian cancer is germ cell cancer, which starts in the cells that form eggs in the ovary. Only about 5 percent of ovarian cancers are germ cell cancer. Finally, stromal cell ovarian cancer begins in the stromal cells, which produce female hormones and form the tissue that holds the ovaries together. Only about 5 percent of all ovarian cancer is stromal cell cancer.

Procedures for detecting, diagnosing, staging, monitoring, prognosticating, preventing, treating, or determining predisposition to cancer, such as breast or ovarian cancer, are of critical importance to the outcome of the patient. For example, patients diagnosed with early breast cancer have greater than a 90% five-year relative survival rate as compared to a survival rate of about 20% for patients diagnosed with distantly metastasized breast cancers. (American Cancer Society statistics). Current prognostic factors such as changes of the p53 tumor suppressor gene, the epidermal growth factor (EGF) receptor, and microvessel density (number of small blood vessels that supply oxygen and nutrition to the cancer), are currently being used and studied for use in helping determine patient survival. Since the prognosis of breast cancer provides criteria for designing optimal therapy and allows for the determination of survival rate of cancer patients, it is advantageous to have novel and independent prognostic factor for breast cancer survival.

Alterations in cadherins, which are calcium-dependent cell-cell adhesion proteins, are understood to be related to tumor development and invasion in carcinomas. The epithelial cadherin, E-cadherin has been found to be a tumor suppressor in the breast (Soler et al. 1999). Low expression of E-cadherin in breast carcinoma has been associated with de-differentiation (Gamallo et al., 1993), increased invasiveness (Sitonen et al., 1996), and high metastatic potential (Hunt et al., 1997). H-cadherin is also believed to have a tumor suppressive role, with a reduced expression in human breast tumors (Lee, 1996). Low expression of P-cadherin in breast carcinoma has also been found to be associated with tumor suppression (Soler et al. 1999).

Deletions or alterations of α-catenins, a cadherin-associated proteins, have been found in a variety of carcinomas, including prostate cancer (Richmond et al, 1997) and breast tumors (Rimm et al., 1995; Shimoyama et al., 1992; Buckholm et al., 1990). Alterations of p120, another cadherin-associated protein, have been shown in, for example, breast cancer (Dillon et al., 1998) bladder cancer (Syrigos et al., 1998a) and gastrointestinal cancer (Karayiannakis et al., 1999). Similarly, altered γ-catenin has been shown to be a factor in bladder cancer (Syrigos et al., 1998b) and thyroid cancer (Bohm et al., 2001). Alterations in β-catenin have also been observed by numerous groups for a variety of carcinomas. β-catenin expression has been studied in colorectal carcinoma (Hugh et al., 1999a, b), melanocytic lesions (Silye et al., 1998) and gastric carcinoma (Jawhari et al., 1997).

Expression of β-catenin has been observed in breast tissue by Inomata et al. (1996) who found that mutations of the APC gene result in truncated APC protein that will not degrade β-catenin. This results in an increase in cellular free β-catenin which was understood to result in the initiation of adenomas. However, it has been previously found that there is no association between any form of β-catenin expression and either tumor stage or tumor grade (Hugh et al., 1999b) and the literature on the alteration, loss, or translocation of β-catenin in carcinomas is inconsistent and confusing. It is therefore an aspect of this invention to provide a method for the prognosis of breast cancer using β-catenin activation.

### BRIEF SUMMARY OF THE INVENTION

The present invention relates to the discovery that activated β-catenin has a negative correlation with and is a strong prognostic factor for survival rates with cancer, including but not limited to breast cancer, ovarian cancer, and colon cancer. In some particularly preferred aspects, the invention relates to breast and/or ovarian cancer. In other specific embodiments, the invention relates to skin cancer, such as pilomatricoma. Activated β-catenin provides independent predictive information on patient survival rate even when other prognostic factors such as lymph node metastasis, tumor size, estrogen receptor, and progesterone receptor status are taken into account. Thus, the inventors envision using β-catenin as a prognostic marker for human cancer.

In some embodiments, the invention relates to method for determining an existence of or characterizing cancer in a subject comprising: obtaining a sample from the subject; and analyzing the sample for an increase or decrease in β-catenin activation. In most preferred embodiments, the methods may be used as a method of determining a prognosis of a subject with cancer. For example, one can determine the prognosis for a patient having a recurrence or metastasis of cancer. The method may, likewise, be used to determine a survival rate of a patient with cancer.

In many preferred embodiments, the cancer is breast cancer or ovarian cancer. However, those of skill in the art, in view of the teachings herein, will recognize that any number of cancers currently known, or determined in the future, that have the same form of β-catenin-involved pathology may be assayed via the methods of the invention. In one embodiment, the cancer relates to having defects in the wnt signaling pathway (Polakis, 2000). For example, it is anticipated that assaying β-catenin activation will allow one to determine a prognosis of a patient with colon cancer. Those of skill will be able to employ the specific teachings regarding breast, ovarian, and colon cancer contained herein to test for the prognostic value of β-catenin for other forms of cancer. In preferred embodiments of the invention, an increase in β-catenin activation indicates a poorer prognosis for the subject, i.e., a decreased or lower subject survival rate. By contrast, in preferred embodiments, an increase in β-catenin activation indicates a better prognosis for the subject, i.e., an increase of higher subject survival rate.

In many embodiments of the invention, the sample comprises a cell. Such cell-containing samples may be obtained by any procedure know to those of skill in the art, including all known biopsy procedures. For example, the sample may be obtained by fine needle aspiration, core needle biopsy, surgical biopsy, vacuum-assisted biopsy, biopsy using an advanced breast biopsy instrument, surgical specimen, analysis of a paraffin embedded tissue, and/or analysis of a frozen tissue imprint.

In cases where the sample is a cell-containing sample, it is possible to determine an increase in β-catenin activation by analyzing subcellular localization of β-catenin at a cellular location. For example, it is possible to look for and/or determine cytoplasmic, nuclear, and/or plasma membrane localization of β-catenin activity or presence in a cell. In some embodiments, a decrease in β-catenin activation is determined by β-catenin localization at a plasma membrane. By contrast, in other embodiments, an increase in β-catenin activation is determined by β-catenin accumulation at a cytoplasm or nucleus. The analysis of β-catenin localization and/or accumulation in a cell may be determined by any of a number of methods for determining localization of proteins in a cell, which are known to those of skill in the art and may be practiced without undue experimentation in view of the teachings of this specification. For example, the analysis may comprise analyzing the sample with a procedure comprising immunohistochemical staining. Alternatively, the analysis may comprise immunoblot analysis. In an alternative embodiment, the cell in the sample may be fractionated by standard techiniques, and the subsequent fractions analyzed for localization of β-catenin.

In some preferred, but certainly non-limiting, embodiments of the invention analyzing the sample, wherein the sample comprises a cell, comprises: transfecting the cell with a cyclin D1 reporter; transfecting the cell with a β-catenin-encoding nucleic acid; and analyzing the transfected cell. For example, the cyclin D1 reporter comprises a Tcf4 mutant reporter. Such methods may further involve the use of antibodies to one or more of β-catenin, cyclin D1 and α-actin. In some specific embodiments, the β-catenin-encoding nucleic acid is further defined as a nucleic acid encoding a β-catenin phosphorylation mutant. In other embodiments, the β-catenin-encoding nucleic acid is myc tagged. Some embodiments of the invention further comprise transfecting the sample with a control vector For example, the control vector can be pcDNA3, as described herein. These methods may further comprise transfecting the sample with a negative β-catenin/Tcf regulator.

In some aspects of the invention, the prognosis, as determined by identifying a degree of β-catenin activation, will determine the course of treatment of a patient with cancer. One can modulate a treatment protocol based on the prognosis determined by the method of the present invention. For example, one may desire to institute a more aggressive treatment protocol in the patient with an increase in β-catenin activation. Those of skill in the art will be able to determine what is a more, or less, aggressive treatment protocol for a given cancer at the time of prognosis. For example, in specific embodiments, a more aggressive treatment comprises administering to the patient a particularly strong or effective chemotherapeutic drug, additional strong or effective chemotherapeutic drug or drugs, or additional anti-cancer treatments such as surgery, radiation, and/or gene therapy.

In some embodiments, a treatment protocol may comprise blocking the β-catenin activation in a subject having an increase in β-catenin activation. In many case, the subject will be a human patient with cancer. Alternatively, the subject may be an animal that serves as a model for an appropriate form of cancer. As discussed above, in some embodiments, the cancer is breast, ovarian, or colon cancer. In some embodiments, the cancer is skin cancer. Blocking of β-catenin activation, may be accomplished by any method known or determined to be useful by those of skill in the art. In some specific embodiments, treatment of the subject will comprise blocking Tcf4 function in the patient having an increase in β-catenin activation. Treatment may comprise, but not be limited to, contacting the subject with an antisense of β-catenin, a dominant-negative mutant of β-catenin, a dominant-negative Tcf4, and/or an antisense of Tcf4. Alternatively, those of skill in the art will be able to determine small molecule, or other chemotherapeutic drugs, that will be of use in the context of the invention. Further, it is possible to determine gene products of a variety of forms that will block β-catenin activation.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A, 1B, 1C, and 1D—Up-regulation of cyclin D1 promoter activity and the protein expression by β-catenin. (A) The 293 cells were transfected with 0.4 μg of cyclin D1 reporter (1745CD1LUC) along with increasing amounts of the wild-type human β-catenin expression plasmid. (B) Cells were transfected with 0.4 μg of cyclin D1 reporter (1745CD1LUC) with 1.0 μg of β-catenin expression plasmid and 1.0 μg of different negative β-catenin/Tcf regulators or with control pcDNA3 plasmid. (C) Cyclin D1 reporters (0.4 μg) (1745CD1LUC, 163CD1LUC, and its mutant, 163CD1LUCm, which had an AT to GC change at nucleotides 75 and 74) and 1.5 μg of β-catenin expression plasmid were transfected into 293 cells. (D) The whole cell lysate of 293 cells, 293 cells transfected with empty vector, and two constitutively-activated β-catenin stable transfectants were separated by SDS/PAGE and analyzed by Western blotting with antibodies that recognized myc-tagged β-catenin, cyclin D1 (purchased from NeoMarkers), and x-actin (purchased from Oncogene).

FIGS. 2A-2B—Correlation between cyclin D1 expression and β-catenin/Tcf4 activity in breast cancer cell lines. (A) Cell lysates from different breast cell lines were separated by SDS/PAGE and analyzed by Western blotting with antibodies, which recognized cyclin D1 and α-actin (Top). The relative β-catenin/Tcf4 activity in different cell lines were determined by the TOP/FOP luciferase activities in each cells (Middle). The density of cyclin D1 bands were quantitated (by NIH IMAGE, an analyzing software) and plotted with β-catenin/Tcf4 activity (TOP/FOP) with the r=0.967 by linear regression. Also, the DNA-binding activities of β-catenin/Tcf4 were determined by gel-shift assay as described previously (Bottom) (Korinek et al., 1997). Lanes 1-8 show the β-catenin/Tcf4-binding activity in indicated cell lines. Lanes 9-12 are the controls to demonstrate the specific binding. Lane 9 and 10, the same as lane 8 except 60-fold excess of wild-type (lane 9) or the mutant (lane 10) cold oligonucleotide was added; lane 11, nuclear extract was from the 293 vector control line; lane 12, nuclear extract was from 293 β-catenin stable line. (B) MCF-7 cells (Top) or HBL100 cells (Bottom) were co-transfected with cyclin D1 reporter (1745CD1LUC) with different negative β-catenin/Tcf regulators or with the control pcDNA3 plasmid. The absolute luciferase activity of cyclin D1 reporter alone in MCF-7 cells was 7-fold higher than that in HBL100 cells.

FIGS. 3A-3B—Correlation between activated p-catenin and cyclin D1 overexpression and their association with poor patient survival rate. (A) Breast cancer tissue stained with β-catenin antibody (a, cytoplasm/nucleus; b, membrane) and cyclin D1 antibody (c, overexpression; d, negative). The right panels (e-h) showed the respective negative controls for a-d using PBS instead of primary antibodies. (B) Kaplan-Meier analysis for survival correlated with the subcellular localization of β-catenin (Top) and cyclin D1 expression (Bottom). The medium of follow-up of patients was 48 months.

FIGS. 4A-4B—Blockage of β-catenin/TCF4 activity inhibited the anchorage-dependant and the anchorage independent growth of MCF-7 cells. (A) Colony formation assay: MCF-7 cells (left) of HBL100 cells (right) were transfected with either a control vector (pcDNA3) or the dominant-negative Tcf4 expression construct. After drug selection, the G418-resistant colonies were stained with crystal violet (top) and quantitated (bottom). (B). Colony formation in soft agar: MCF-7 cells without transfection or transiently transfected with either pCDNA or dnTcf4 constructs were plated in semi-solid medium containing 0.5% agarose over a 1% layer. After three weeks, the colonies were stained by p-iodonitrotetrazolium violet and scored.

### DETAILED DESCRIPTION OF THE INVENTION

### I. DEFINITIONS

As used herein the specification, “a” or “an” may mean one or more. As used herein in the claim(s), when used in conjunction with the word “comprising”, the words “a” or “an” may mean one or more than one. As used herein “another” may mean at least a second or more.

The terms “isolated” “purified” or “biologically pure” refer to material that is substantially or essentially free from components which normally accompany it as found in its native state. Purity and homogeneity are typically determined using analytical chemistry techniques such as polyacrylamide gel electrophoresis or high-performance liquid chromatography. A protein that is the predominant species present in a preparation is substantially purified. In particular, an isolated β-catenin or cyclin D1 nucleic acid is separated, e.g., from open reading frames or fragments of open reading frames, e.g., that naturally flank the gene and encode proteins other than β-catenin or cyclin D1 protein. An isolated β-catenin or cyclin D1 nucleic acid is typically contiguous, i.e., heterologous sequences are typically not embedded in the β-catenin nucleic acid sequence, although heterologous sequences are often found adjoining an isolated β-catenin or cyclin D1 nucleic acid sequence. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

As used herein, “mutant” refers to a change in the sequence of a nucleic acid or its encoded protein, polypeptide, or peptide that is the result of recombinant DNA technology.

As used herein “wild-type” refers to the naturally occurring sequence of a nucleic acid at a genetic locus in the genome of an organism, and sequences transcribed or translated from such a nucleic acid. Thus, the term “wild-type” also may refer to the amino acid sequence encoded by the nucleic acid. As a genetic locus may have more than one sequence or alleles in a population of individuals, the term “wild-type” encompasses all such naturally occurring alleles.

### II. THE PRESENT INVENTION

The present invention provides a novel and powerful prognostic marker for cancer, including breast, ovarian, and colon cancers. β-catenin, which can function as an oncogene when it is translocated to the nucleus, binds to T cell factor or lymphoid enhancer factor family members, and transactivates its target genes. In this invention, it was determined that cyclin D1 is one of the targets of β-catenin in cancer cells. Transactivation of β-catenin correlated significantly with cyclin D1 expression. High β-catenin activity significantly correlated with poor prognosis of the patients and is a strong and independent prognostic factor in cancer. Moreover, activated β-catenin is a strong prognostic factor that provided additional and independent predictive information on patient survival rate.

In specific embodiments of the present invention, specific nucleic acid and amino acid sequences are utilized for methods and/or compositions described herein. Although a skilled artisan is aware how to retrieve such sequences from publicly available databases such as the National Center for Biotechonology Information's GenBank database, specific exemplary sequences are herein provided. Examples of β-catenin amino acid sequences, followed by their GenBank accession number, include SEQ ID NO: 4 (AAD32267); SEQ ID NO: 5 (CAA61107); SEQ ID NO: 6 (CAA79497); SEQ ID NO: 7 (A38973); SEQ ID NO: 8 (S35091) (mouse); and SEQ ID NO: 9 (AAD28504). Examples of β-catenin nucleic acid sequences include SEQ ID NO: 10 (X87838); SEQ ID NO: 11 (X89448); SEQ ID NO: 12 (Z19054); SEQ ID NO: 13 (AF397179) (rat); SEQ ID NO: 14 (NM_{—}053357) (rat); and SEQ ID NO: 15 (NM_{—}007614) (mouse). An example of a hTCF-4 nucleic acid sequence is comprised in SEQ ID NO: 16 (Y11306). An example of a hTCF-4 amino acid sequence is in SEQ ID NO: 17 (CAA72166). Examples of cyclin D1 gene promoter regions include SEQ ID NO: 18 (Z29078), SEQ ID NO: 19 (AF148946) (rat) and SEQ ID NO: 20 (AF182716) (mouse). Examples of cyclin D1 nucleic acid sequences include SEQ ID NO: 21 (D14014) and SEQ ID NO: 22 (NM_{—}007631) (mouse). Examples of cyclin D1 amino acid sequences include SEQ ID NO: 23 (AAH25302); SEQ ID NO: 24 (NP_{—}444284); SEQ ID NO: 25 (P24385); SEQ ID NO: 26 (NP_{—}031657) (mouse); SEQ ID NO: 27 (CAA54800); SEQ ID NO: 28 (AAH00076); SEQ ID NO: 29 (A38977); and SEQ ID NO: 30 (AAF23491) (mouse).

### III. β-CATENIN

β-catenin is a 92-kd protein, initially identified as a cell-cell adhesion molecule. Recent studies have indicated that β-catenin can also be translocated to the nucleus and transactivate genes whose functions are implicated in cancer formation and progression. In the past, the β-catenin pathway has been studied mainly in colon carcinoma. In 85% of colorectal cancers, the tumor suppressor adenomatous polyposis coli (“APC”) gene is lost or inactivated. Inactivation of the APC gene leads to β-catenin accumulation in the nucleus and, presumably, stimulation of tumor cell growth. Wnt signaling has now been linked to activation of the c-MYC oncogene (Pennisi, 1998). Almost 100% of colon cancers have either mutated β-catenin or deleted APC, which is expected to activate the β-catenin pathway. Barker et al. recently determined that hTcf-4 binds to β-catenin and activates transcription in colorectal epithelial cells (U.S. Pat. No. 5,998,600). Two groups identified cyclin D1 as the β-catenin target in colon carcinoma (Tetsu et al., 1999; Shtutman et al., 1999). However, it is worthwhile to mention that cyclin D1 overexpression has been found in only 30% of colon cancer ( Bartkova et al., 1994; Arber et al., 1996), which might not be consistent with almost 100% deregulation of the β-catenin pathway, suggesting that the overexpression of cyclin D1 in colon cancer may be more complicated than purely up-regulating by β-catenin.

β-catenin was first isolated as a cell-cell adhesion protein that associated with the intracellular domain of E-cadherin, a component of the adhesion junction in epithelial cells (Aberle, 1996). However, in addition to serve as an adhesion molecule, β-catenin has been shown to transduce the signals along the Wnt pathway (Fasgotto et al., 1996; Sanson et al., 1996). The transcriptional activation of target genes in response to Wnt signaling is dependent on the nuclear translocation of free cytoplasmic β-catenin and complex formation with a member of the Tcf/Lef architectural transcription factor. The regulation of this transcriptional activity is mainly achieved by strictly controlling the levels of free cytoplasmic β-catenin available for binding to the Tcf/Lef. In the absence of a Wnt signal, a quaternary cytoplasmic complex comprising β-catenin, adenomatous polyposis coli (APC), Conduction/Axin, and GSK3β mediates the phosphorylation and consequently the targeted destruction of β-catenin via the ubiquitin-proteasome pathway (Polakis, 1999). Mutation of APC in colon carcinoma or the mutations of β-catenin in a variety of cancer types could both prevent the down-regulation of β-catenin and cause constitutively activated β-catenin signaling, which contributes to the oncogenesis process effect of those cancers (Rubinfeld et al., 1997; Korinek et al., 1997; Polakis, 1999).

Mutations of APC or β-catenin in colon carcinoma cells have been found by He et al. (U.S. Pat. No. 6,140,052) and Barker (U.S. Pat. No. 5,998,600). So far, no mutation of APC or β-catenin have been found in breast cancer. However, many studies have indicated a possible role for the Wnt pathway in breast cancer. For example, mouse Wnt1, Wnt3 and Wnt10b have been found to be among the oncogenes activated by the insertion of MMTV (Musse et al., 1984; Roelink et al., 1990). Mammary hyperplasias have also occurred in Wnt1 transgenic mice (Tsukamoto et al., 1988). In addition, several members of the Wnt family have been shown to induce cell proliferation (Blasband et al., 1992; Wang et al., 1994). Moreover, the expression of different Wnt members has been reported to correlate with abnormal cell proliferation in human breast tissue, suggesting the possible involvement of Wnt and the β-catenin pathway in breast cancer (Dale et al., 1996; Lejeune e al., 1995; Bui et al., 1997).

### IV. CYCLIN D1

Cyclin D1 is one of the targets for β-catenin in breast cancer. More importantly, it is demonstrated to have a significant role regarding activated β-catenin in breast cancer both by molecular studies in cell culture and by clinical studies on breast tumor samples. Consistent with these findings, the studies provide strong evidence supporting the biological significance and clinical relevance of this pathway in human breast cancer. In contrast to colon carcinoma, the strong correlation between β-catenin activity and cyclin D1 expression was found in both breast cancer cell lines and breast patient tissue samples. Thus, the data presented herein opens a new direction in the research of breast cancer involving both cancer formation and progression and provides an opportunity for development of potential therapy by at least blocking the β-catenin/Tcf4 pathway in breast cancer cells.

Cyclin D1 overexpression has been found in 50% of patients with breast cancer (Gillet et al., 1994); Bartkova, et al, 1994), whereas gene amplification accounted for only 15-20% of these cases (Fantl et al., 1993). Therefore, other mechanisms such as up-regulation of gene transcription must have played a substantial role in the overexpression of cyclin D1. By analyzing the promoter region of cyclin D1, a perfect T cell factor 4 (Tcf4)-binding site (CTTTGATC; SEQ ID NO: 1) located between nucleotides 80 and 73 was identified, suggesting the potential involvement of the β-catenin/Tcf4 pathway in the regulation of cyclin D1 expression.

Several mechanisms have been reported to cause the deregulation in cyclin D1 expression, including deletion of the adenomatous polyposis coli (APC) gene, mutation of β-catenin, and activation of the Wnt pathway (Polakis, 1999). Although deletion of APC and mutation of β-catenin have been found in many types of cancers (Polakis, 1999), so far no such defects have been reported in breast cancer. However, many studies have indicated a possible role for the Wnt pathway in breast cancer. For example, mouse Wnt1, Wnt3, and Wnt10b have been found to be among the oncogenes activated by the insertion of mouse mammary tumor virus (MMTV) (Nusse et al., 1984; Roelink et al., 1990)). Mammary hyperplasias also have occurred in Wnt1 transgenic mice (Tsukamoto et al., 1988). In addition, several members of the Wnt family have been shown to induce cell proliferation (Blasband et al., 1992; Wong et al., 1994) ). Moreover, the expression of different Wnt members has been reported to correlate with abnormal cell proliferation in human breast tissue, suggesting the possible involvement of Wnt and the β-catenin pathway in breast cancer (Dale et al., 1996; Lejeune et al., 1995; Bui et al., 1997).

### V. CANCER TYPES

Beta-catenin can be used as a prognostic factor for cancer. It is conceived that β-catenin be used as a prognostic factor for a variety of types of cancer such as breast cancer, colorectal cancer, thyroid cancer, brain cancer, head and neck cancer, prostate, liver, myelomas, bladder, blood, bone, bone marrow, esophagus, gastrointestine, kidney, lung, nasopharynx, ovary, skin, stomach, and uterus cancers. In some embodiments, it is a prognostic factor for breast cancer. It is preferred that the activation of β-catenin be used as a prognostic factor for breast cancer. There are numerous types of breast cancers for which patient survival rate can be determined with a method of the current invention.

### A. Ductal Carcinoma in situ (DCIS)

Ductal carcinoma in situ (also known as intraductal carcinoma) is the most common type of noninvasive breast cancer. Cancer cells are inside the ducts but have not spread through the walls of the ducts into the fatty tissue of the breast. DCIS is subclassified based on its grade and type, in order to help predict the risk of cancer returning after treatment and to help select the most appropriate treatment. There are several types of DCIS, but the most important distinction among them is whether or not tumor cell necrosis (areas of dead or degenerating cancer cells) is present. The term comedocarcinoma is often used to describe a type of DCIS with necrosis.

### B. Infiltrating (or Invasive) Ductal Carcinoma (IDC)

Starting in a milk passage, or duct, of the breast, this cancer has broken through the wall of the duct and invades the fatty tissue of the breast. At this point, it has the potential to metastasize, or spread, to other parts of the body through the lymphatic system and bloodstream. Infiltrating ductal carcinoma accounts for about 80% of invasive breast cancers, and the survival rate can be determined by β-catenin activation.

### C. Infiltrating (or Invasive) Lobular Carcinoma (ILC)

ILC starts in the milk-producing glands, and, similar to IDC, has the potential to metastasize. About 10% to 15% of invasive breast cancers are invasive lobular carcinomas. ILC may be more difficult to detect by mammogram than IDC.

### D. Inflammatory Breast Cancer

This is a relatively rare type of invasive breast cancer which accounts for about 1% of all breast cancers. Inflammatory breast cancer makes the skin of the breast look red and feel warm, as if it was infected and inflamed. The skin has a thick, pitted appearance that doctors often describe as resembling an orange peel. Sometimes the skin develops ridges and small bumps that look like hives. Cancer cells blocking lymph vessels or channels in the skin over the breast cause these symptoms.

### E. Lobular Carcinoma in situ (LCIS)

While not a true cancer, LCIS (also called lobular neoplasia) is sometimes classified as a type of noninvasive breast cancer. LCIS begins in the milk-producing glands, but does not penetrate through the wall of the lobules. It is understood that LCIS does not become an invasive cancer, but women with this condition do have a higher risk of developing an invasive breast cancer. The determination of β-catenin activation can be used in LCIS to help determine the survival rate.

### F. Medullary Carcinoma

This special type of infiltrating breast cancer has a relatively well defined, distinct boundary between tumor tissue and normal tissue. It also has some other special features, including the large size of the cancer cells and the presence of immune system cells at the edges of the tumor. Medullary carcinoma accounts for about 5% of breast cancers. The outlook, or prognosis, for this kind of breast cancer is better than for other types of invasive breast cancer.

### G. Mucinous Carcinoma

This rare type of invasive breast cancer is formed by mucus-producing cancer cells. The prognosis for mucinous carcinoma is better than for the more common types of invasive breast cancer. Colloid carcinoma is another name for this type of breast cancer.

### H. Paget's Disease of the Nipple

This type of breast cancer starts in the breast ducts and spreads to the skin of the nipple and then to the areola, the dark circle around the nipple. It is a rare type of breast cancer, occurring in only 1% of all cases. The skin of the nipple and areola often appears crusted, scaly, and red, with areas of bleeding or oozing. The woman may notice burning or itching. Paget's disease may be associated with in situ carcinoma, or with infiltrating breast carcinoma.

### I. Phyllodes Tumor

This very rare type of breast tumor forms from the stroma (connective tissue) of the breast, in contrast to carcinomas which develop in the ducts or lobules. Phyllodes, or phylloides tumors are usually benign but on rare occasions may be malignant (having the potential to metastasize). Benign phyllodes tumors are successfully treated by removing the mass and a narrow margin of normal breast tissue. A malignant phyllodes tumor is treated by removing it along with a wider margin of normal tissue, or by mastectomy. These cancers do not respond to hormonal therapy and are not so likely to respond to chemotherapy or radiation therapy. Both benign and malignant phyllodes tumors are also referred to as cystosarcoma phyllodes.

### J. Tubular Carcinoma

Accounting for about 2% of all breast cancers, tubular carcinomas are a special type of infiltrating breast carcinoma. They have a better prognosis than usual infiltrating ductal or lobular carcinomas. (American Cancer Society Web page). For this and other types of breast cancer, the determination of β-catenin activation can be used to help determine whether a patient is at risk for the development, recurrence, or metastasis of cancer.

### VI. METHODS OF GENE TRANSFER

In order to determine β-catenin activation, it will be necessary to determine β-catenin expression in tumor tissues, which is preferably done by immunohistochemical staining. This section provides a discussion of methods and compositions of gene or nucleic acid transfer, including transfer of antisense sequences that can be used in the present invention to transfer the expression constructs of the present invention into a cell.

In order to determine β-catenin activation, it will be necessary to transfer the expression constructs of the present invention into a cell, such as a cell from a patient sample tissue. This section provides a discussion of methods and compositions of gene or nucleic acid transfer, including transfer of antisense sequences.

The mammalian cyclin D1 genes are incorporated into an adenoviral infectious particle to mediate gene transfer to a cell. Additional expression constructs encoding other therapeutic agents as described herein may also be transferred via viral transduction using infectious viral particles, for example, by transformation with an adenovirus vector of the present invention as described herein below. Alternatively, retroviral or bovine papilloma virus may be employed, both of which permit permanent transformation of a host cell with a gene(s) of interest. Thus, in one example, viral infection of cells is used in order to deliver therapeutically significant genes to a cell. Typically, the virus simply will be exposed to the appropriate host cell under physiologic conditions, permitting uptake of the virus. Though adenovirus is exemplified, the present methods may be advantageously employed with other viral vectors, as discussed below.

### A. Adenoviral Vectors

A particular method for delivery of the expression constructs for the determination of β-catenin activation involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and/or (b) to ultimately express a tissue and/or cell-specific construct that has been cloned therein.

The expression vector comprises a genetically engineered form of adenovirus. Knowledge of the genetic organization and/or adenovirus, a 36 kb, linear, double-stranded DNA virus, allows substitution of large pieces of adenoviral DNA with foreign sequences up to 7 kb (Grunhaus and/or Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and/or no genome rearrangement has been detected after extensive amplification.

Adenovirus is particularly suitable for use as a gene transfer vector because of its mid-sized genome, ease of manipulation, high titer, wide target-cell range and/or high infectivity. Both ends of the viral genome contain 100-200 base pair inverted repeats (ITRs), which are cis elements necessary for viral DNA replication and/or packaging. The early (E) and/or late (L) regions of the genome contain different transcription units that are divided by the onset of viral DNA replication. The E1 region (E1A and/or E1B) encodes proteins responsible for the regulation of transcription of the viral genome and/or a few cellular genes. The expression of the E2 region (E2A and/or E2B) results in the synthesis of the proteins for viral DNA replication. These proteins are involved in DNA replication, late gene expression and/or host cell shut-off (Renan, 1990). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and/or all the mRNAs issued from this promoter possess a 5′-tripartite leader (TPL) sequence which makes them preferred mRNA's for translation.

In a current system, recombinant adenovirus is generated from homologous recombination between shuttle vector and/or provirus vector. Due to the possible recombination between two proviral vectors, wild-type adenovirus may be generated from this process. Therefore, it is critical to isolate a single clone of virus from an individual plaque and/or examine its genomic structure.

Generation and/or propagation of the current adenovirus vectors, which are replication deficient, depend on a unique helper cell line, designated 293, which was transformed from human embryonic kidney cells by Ad5 DNA fragments and/or constitutively expresses E1 proteins (E1A and/or E1B; Graham et al., 1977). Since the E3 region is dispensable from the adenovirus genome (Jones and/or Shenk, 1978), the current adenovirus vectors, with the help of 293 cells, carry foreign DNA in either the E1, the D3 and/or both regions (Graham and/or Prevec, 1991). Recently, adenoviral vectors comprising deletions in the E4 region have been described (U.S. Pat. No. 5,670,488, incorporated herein by reference).

In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and/or E3 regions, the maximum capacity of the current adenovirus vector is under 7.5 kb, and/or about 15% of the total length of the vector. More than 80% of the adenovirus viral genome remains in the vector backbone.

Helper cell lines may be derived from human cells such as embryonic kidney cells, muscle cells, hematopoietic cells and/or other embryonic mesenchymal and/or epithelial cells. Alternatively, the helper cells may be derived from the cells of other mammalian species that are permissive for human adenovirus. Such cells include, e.g., Vero cells and/or other monkey embryonic mesenchymal and/or epithelial cells.

Recently, Racher et al. (1995) disclosed improved methods for culturing 293 cells and/or propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and/or left stationary, with occasional agitation, for 1 to 4 h. The medium is then replaced with 50 ml of fresh medium and/or shaking initiated. For virus production, cells are allowed to grow to about 80% confluence, after which time the medium is replaced (to 25% of the final volume) and/or adenovirus added at an MOI of 0.05. Cultures are left stationary overnight, following which the volume is increased to 100% and/or shaking commenced for another 72 h.

Other than the requirement that the adenovirus vector be replication defective, and/or at least conditionally defective, the nature of the adenovirus vector is not believed to be crucial to the successful practice of the invention. The adenovirus may be of any of the 42 different known serotypes and/or subgroups A-F. Adenovirus type 5 of subgroup C is the preferred starting material in order to obtain the conditional replication-defective adenovirus vector for use in the present invention. This is because Adenovirus type 5 is a adenovirus about which a great deal of biochemical and/or genetic information is known, and/or it has historically been used for most constructions employing adenovirus as a vector.

As stated above, the typical vector according to the present invention is replication defective and/or will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) and/or in the E4 region where a helper cell line and/or helper virus complements the E4 defect.

Adenovirus growth and/or manipulation is known to those of skill in the art, and/or exhibits broad host range in vitro and/or in vivo. This group of viruses can be obtained in high titers, e.g., 10⁹ to 10¹¹ plaque-forming units per ml, and/or they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and/or therapeutic potential as in vivo gene transfer vectors.

Adenovirus vectors have been used in eukaryotic gene expression (Levrero et al., 1991; Gomez-Foix et al., 1992) and/or vaccine development (Grunhaus and/or Horwitz, 1992; Graham and/or Prevec, 1992). Recently, animal studies suggested that recombinant adenovirus could be used for gene therapy (Stratford-Perricaudet and/or Perricaudet, 1991a; Stratford-Perricaudet et al., 1991b; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and/or Gerard, 1993) and/or stereotactic inoculation into the brain (Le Gal La Salle et al., 1993). Recombinant adenovirus and/or adeno-associated virus (see below) can both infect and/or transduce non-dividing hyman primary cells.

### B. AAV Vectors

Adeno-associated virus (AAV) is an attractive vector system for use in the cell transduction of the present invention as it has a high frequency of integration and/or it can infect nondividing cells, thus making it useful for delivery of genes into mammalian cells, for example, in tissue culture (Muzyczka, 1992) and/or in vivo. AAV has a broad host range for infectivity (Tratschin et al., 1984; Laughlin et al., 1986; Lebkowski et al., 1988; McLaughlin et al., 1988). Details concerning the generation and/or use of rAAV vectors are described in U.S. Pat. No. 5,139,941 and/or U.S. Pat. No. 4,797,368, each incorporated herein by reference.

Studies demonstrating the use of AAV in gene delivery include LaFace et al. (1988); Zhou et al. (1993); Flotte et al. (1993); and/or Walsh et al. (1994). Recombinant AAV vectors have been used successfully for in vitro and/or in vivo transduction of marker genes (Kaplitt et al., 1994; Lebkowski et al., 1988; Samulski et al., 1989; Yoder et al., 1994; Zhou et al., 1994; Hermonat and/or Muzyczka, 1984; Tratschin et al., 1985; McLaughlin et al., 1988) and/or genes involved in human diseases (Flotte et al., 1992; Luo et al., 1994; Ohi et al., 1990; Walsh et al., 1994; Wei et al., 1994).

AAV is a dependent parvovirus in that it requires coinfection with another virus (either adenovirus and/or a member of the herpes virus family) to undergo a productive infection in cultured cells (Muzyczka, 1992). In the absence of coinfection with helper virus, the wild type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and/or Smith, 1994). When a cell carrying an AAV provirus is superinfected with a helper virus, the AAV genome is “rescued” from the chromosome and/or from a recombinant plasmid, and/or a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and/or an expression plasmid containing the wild type AAV coding sequences without the terminal repeats, for example pIM45 (McCarty et al., 1991; incorporated herein by reference). The cells are also infected and/or transfected with adenovirus and/or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions and/or cell lines containing the AAV coding regions and/or some and/or all of the adenovirus helper genes could be used (Yang et al., 1994; Clark et al., 1995). Cell lines carrying the rAAV DNA as an integrated provirus can also be used (Flotte et al., 1995).

### C. Retroviral Vectors

Retroviruses can be gene delivery vectors due to their ability to integrate their genes into the host genome, transferring a large amount of foreign genetic material, infecting a broad spectrum of species and/or cell types and/or of being packaged in special cell-lines (Miller, 1992).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and/or directs synthesis of viral proteins. The integration results in the retention of the viral gene sequences in the recipient cell and/or its descendants. The retroviral genome contains three genes, gag, pol, and/or env that code for capsid proteins, polymerase enzyme, and/or envelope components, respectively. A sequence found upstream from the gag gene contains a signal for packaging of the genome into virions. Two long terminal repeat (LTR) sequences are present at the 5′ and/or 3′ ends of the viral genome. These contain strong promoter and/or enhancer sequences and/or are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and/or env genes but without the LTR and/or packaging components is constructed (Mann et al., 1983). When a recombinant plasmid containing a cDNA, together with the retroviral LTR and/or packaging sequences is introduced into this cell line (by calcium phosphate precipitation for example), the packaging sequence allows the RNA transcript of the recombinant plasmid to be packaged into viral particles, which are then secreted into the culture media (Nicolas and/or Rubenstein, 1988; Temin, 1986; Mann et al., 1983). The media containing the recombinant retroviruses is then collected, optionally concentrated, and/or used for gene transfer. Retroviral vectors are able to infect a broad variety of cell types. However, integration and/or stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Gene delivery using second generation retroviral vectors has been reported. Kasahara et al. (1994) prepared an engineered variant of the Moloney murine leukemia virus, that normally infects only mouse cells, and/or modified an envelope protein so that the virus specifically bound to, and/or infected cells bearing the erythropoietin (EPO) receptor. This was achieved by inserting a portion of the EPO sequence into an envelope protein to create a chimeric protein with a new binding specificity.

### D. Herpesvirus

Because herpes simplex virus (HSV) is neurotropic, it has generated considerable interest in treating nervous system disorders. Moreover, the ability of HSV to establish latent infections in non-dividing neuronal cells without integrating in to the host cell chromosome or otherwise altering the host cell's metabolism, along with the existence of a promoter that is active during latency makes HSV an attractive vector. And though much attention has focused on the neurotropic applications of HSV, this vector also can be exploited for other tissues given its wide host range.

Another factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations.

HSV also is relatively easy to manipulate and can be grown to high titers. Thus, delivery is less of a problem, both in terms of volumes needed to attain sufficient MOI and in a lessened need for repeat dosings. For a review of HSV as a gene therapy vector, see (Glorioso et al., 1995).

HSV, designated with subtypes 1 and 2, are enveloped viruses that are among the most common infectious agents encountered by humans, infecting millions of human subjects worldwide. The large, complex, double-stranded DNA genome encodes for dozens of different gene products, some of which derive from spliced transcripts. In addition to virion and envelope structural components, the virus encodes numerous other proteins including a protease, a ribonucleotide reductase, a DNA polymerase, a ssDNA binding protein, a helicase/primase, a DNA dependent ATPase, dUTPase and others.

HSV genes from several groups whose expression is coordinately regulated and sequentially ordered in a cascade fashion (Honess and Roizman, 1974; Honess and Roizman, 1975; Roizman and Sears, 1995). The expression of α genes, the first set of genes to be expressed after infection, is enhanced by the virion protein number 16, or α-transducing factor (Post et al., 1981; Batterson and Roizman, 1983; Campbell et al., 1983). The expression of β genes requires functional a gene products, most notably ICP4, which is encoded by the α4 gene (DeLuca et al., 1985). γ genes, a heterogeneous group of genes encoding largely virion structural proteins, require the onset of viral DNA synthesis for optimal expression (Holland et al., 1980).

In line with the complexity of the genome, the life cycle of HSV is quite involved. In addition to the lytic cycle, which results in synthesis of virus particles and, eventually, cell death, the virus has the capability to enter a latent state in which the genome is maintained in neural ganglia until some as of yet undefined signal triggers a recurrence of the lytic cycle. Avirulent variants of HSV have been developed and are readily available for use in gene therapy contexts (U.S. Pat. No. 5,672,344).

### E. Lentiviral Vectors

Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. The lentiviral genome and the proviral DNA have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′ LTR's serve to promote transcription and polyadenylation of the virion RNA's. The LTR contains all other cis-acting sequences necessary for viral replication. Lentiviruses have additional genes including vif vpr, tat, rev, vpu, nef and vpx.

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA. However, the resulting mutant remains capable of directing the synthesis of all virion proteins.

Lentiviral vectors are known in the art, see Naldini et al., (1996); Zufferey et al., (1997), U.S. Pat. Nos. 6,013,516 and 5,994,136. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest.

Recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. This describes a first vector that can provide a nucleic acid encoding a viral gag and a pol gene and another vector that can provide a nucleic acid encoding a viral env to produce a packaging cell. Introducing a vector providing a heterologous gene into that packaging cell yields a producer cell which releases infectious viral particles carrying the foreign gene of interest. The env preferably is an amphotropic envelope protein which allows transduction of cells of human and other species.

One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

The vector providing the viral env nucleic acid sequence is associated operably with regulatory sequences, e.g., a promoter or enhancer. The regulatory sequence can be any eukaryotic promoter or enhancer, including for example, the Moloney murine leukemia virus promoter-enhancer element, the human cytomegalovirus enhancer or the vaccinia P7.5 promoter. In some cases, such as the Moloney murine leukemia virus promoter-enhancer element, the promoter-enhancer elements are located within or adjacent to the LTR sequences.

The heterologous or foreign nucleic acid sequence is linked operably to a regulatory nucleic acid sequence. Preferably, the heterologous sequence is linked to a promoter, resulting in a chimeric gene. The heterologous nucleic acid sequence may also be under control of either the viral LTR promoter-enhancer signals or of an internal promoter, and retained signals within the retroviral LTR can still bring about efficient expression of the transgene. Marker genes may be utilized to assay for the presence of the vector, and thus, to confirm infection and integration. The presence of a marker gene ensures the selection and growth of only those host cells which express the inserts. Typical selection genes encode proteins that confer resistance to antibiotics and other toxic substances, e.g., histidinol, puromycin, hygromycin, neomycin, methotrexate, etc. and cell surface markers.

The vectors are introduced via transfection or infection into the packaging cell line. The packaging cell line produces viral particles that contain the vector genome. Methods for transfection or infection are well known by those of skill in the art. After cotransfection of the packaging vectors and the transfer vector to the packaging cell line, the recombinant virus is recovered from the culture media and titered by standard methods used by those of skill in the art. Thus, the packaging constructs can be introduced into human cell lines by calcium phosphate transfection, lipofection or electroporation, generally together with a dominant selectable marker, such as neo, DHFR, Gln synthetase or ADA, followed by selection in the presence of the appropriate drug and isolation of clones. The selectable marker gene can be linked physically to the packaging genes in the construct.

### F. Vaccinia Virus

Vaccinia virus vectors have been used extensively because of the ease of their construction, relatively high levels of expression obtained, wide host range and large capacity for carrying DNA. Vaccinia contains a linear, double-stranded DNA genome of about 186 kb that exhibits a marked “A-T” preference. Inverted terminal repeats of about 10.5 kb flank the genome. The majority of essential genes appear to map within the central region, which is most highly conserved among poxviruses. Estimated open reading frames in vaccinia virus number from 150 to 200. Although both strands are coding, extensive overlap of reading frames is not common.

At least 25 kb can be inserted into the vaccinia virus genome (Smith and Moss, 1983). Prototypical vaccinia vectors contain transgenes inserted into the viral thymidine kinase gene via homologous recombination. Vectors are selected on the basis of a tk-phenotype. Inclusion of the untranslated leader sequence of encephalomyocarditis virus, the level of expression is higher than that of conventional vectors, with the transgenes accumulating at 10% or more of the infected cell's protein in 24 h (Elroy-Stein et al., 1989).

### G. Polyoma Viruses

The empty capsids of papovaviruses, such as the mouse polyoma virus, have received attention as possible vectors for gene transfer (Barr et al., 1979), first described the use of polyoma empty when polyoma DNA and purified empty capsids were incubated in a cell-free system. The DNA of the new particle was protected from the action of pancreatic DNase. Slilaty and Aposhian (1983) described the use of those reconstituted particles for transferring a transforming polyoma DNA fragment to rat FIII cells. The empty capsids and reconstituted particles consist of all three of the polyoma capsid antigens VP1, VP2 and VP3 and there is no suggestion that pseudocapsids consisting of only the major capsid antigen VP1, could be used in genetic transfer.

(Montross et al., 1991), described only the major capsid antigen, the cloning of the polyoma virus VP1 gene and its expression in insect cells. Self-assembly of empty pseudocapsids consisting of VP1 is disclosed, and pseudocapsids are said not to contain DNA. It is also reported that DNA inhibits the in vitro assembly of VP 1 into empty pseudocapsids, which suggests that said pseudocapsids could not be used to package exogenous DNA for transfer to host cells. The results of (Sandig et al., 1993), showed that empty capsids incorporating exogenous DNA could transfer DNA in a biologically functional manner to host cells only if the particles consisted of all three polyoma capsid antigens VP1, VP2 and VP3. Pseudocapsids consisting of VP1 were said to be unable to transfer to exogenous DNA so that it could be expressed in the host cells, probably due the absence of Ca²⁺ ions in the medium in which the pseudocapsids were prepared. Haynes et al. (1993) discuss the effect of calcium ions on empty VP1 pseudocapsid assembly.

U.S. Pat. No. 6,046,173, issued on Apr. 4, 2000, and entitled “Polyoma virus pseudocapsids and method to deliver material into cell,” reports on the use of a pseudocapsid formed from papovavirus major capsid antigen and excluding minor capsid antigens, which pseudocapsid incorporates exogenous material for gene transfer.

### H. Other Viral Vectors

Other viral vectors may be employed as expression constructs in the present invention. Vectors derived from viruses such as sindbis virus and/or cytomegalovirus. They offer several attractive features for various mammalian cells (Friedmann, 1989; Ridgeway, 1988; Baichwal and/or Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and/or reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and/or pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

### I. Modified Viruses

In still further embodiments of the present invention, the nucleic acids to be delivered are housed within an infective virus that has been engineered to express a specific binding ligand. The virus particle will thus bind specifically to the cognate receptors of the target cell and/or deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and/or against a specific cell receptor were used. The antibodies were coupled via the biotin components by using streptavidin (Roux et al., 1989). Using antibodies against major histocompatibility complex class I and/or class II antigens, they demonstrated the infection of a variety of human cells that bore those surface antigens with an ecotropic virus in vitro (Roux et al., 1989).

### VII. Assays for β-Catenin Activation

In still further embodiments, the present invention provides methods for identifying whether the activation of β-catenin has been altered. The changes in β-catenin activation can be determined by observing the localization of β-catenin at different locations in the cell. β-catenin localization at the cell cytoplasm or cell nucleus is described as activation of β-catenin where localization of β-catenin at the plasma membrane is described as a decrease in β-catenin activation. It is contemplated that a variety of techniques can be used to obtain β-catenin activation.

### A. Immunoassays

Immunodetection methods may be used in the current invention for detecting, binding, purifying, removing and quantifying the proteins and peptides of the current invention. The proteins or peptides of the present invention may be employed to detect antibodies having reactivity therewith, or, alternatively, antibodies prepared in accordance with the present invention, may be employed to detect activation of β-catenin.

The steps of various useful immunodetection methods have been described in the scientific literature, such as, e.g., Nakamura et al. (1987; incorporated herein by reference). Immunoassays, in their most simple and direct sense, are binding assays. Certain preferred immunoassays are the various types of enzyme linked immunosorbent assays (ELISAs), radioimmunoassays (RIA) and immunobead capture assay. Immunohistochemical detection using tissue sections also is particularly useful. However, it will be readily appreciated that detection is not limited to such techniques, and Western blotting, dot blotting, FACS analyses, and the like also may be used in connection with the present invention.

In general, immunobinding methods include obtaining a sample suspected of containing a protein, peptide or antibody, and contacting the sample with an antibody or protein or peptide in accordance with the present invention, as the case may be, under conditions effective to allow the formation of immunocomplexes.

The immunobinding methods of this invention include methods for detecting or quantifying the amount of a reactive component in a sample, which methods require the detection or quantitation of any immune complexes formed during the binding process. Here, one would obtain a β-catenin protein, peptide or a corresponding antibody, and contact it with an antibody or protein or peptide, as the case may be, and then detect or quantify the amount of immune complexes formed under the specific conditions.

In terms of antigen detection, the biological sample analyzed may be any sample that is suspected of containing an antigen specific to the cell adhesion proteins or cyclin D1 of the current invention. The sample can be a tissue section or specimen, a homogenized tissue extract, an isolated cell, a cell membrane preparation, separated or purified forms of any of the above protein-containing compositions, or even any biological fluid that comes into contact with tissue such as blood. Contacting the chosen biological sample with the protein, peptide or antibody under conditions effective and for a period of time sufficient to allow the formation of immune complexes (primary immune complexes) is generally a matter of simply adding the composition to the sample and incubating the mixture for a period of time long enough for the antibodies to form immune complexes with, i.e., to bind to, any antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any radioactive, fluorescent, biological or enzymatic tags or labels of standard use in the art. U.S. Pat. Nos. concerning the use of such labels include 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody or a biotin/avidin ligand binding arrangement, as is known in the art.

The protein, peptide or corresponding antibody employed in the detection may itself be linked to a detectable label, wherein one would then simply detect this label, thereby allowing the amount of the primary immune complexes in the composition to be determined. Epitope tags are useful for the labeling and detection of proteins using immunoblotting, immunoprecipitation and immunostaining techniques. Due to their small size, they are unlikely to affect the tagged protein's biochemical properties. The Myc epitope tag is widely used to detect expression of recombinant proteins in bacteria, yeast, insect and mammalian cell systems (Munro et al, 1984).

Alternatively, the first added component that becomes bound within the primary immune complexes may be detected by means of a second binding ligand that has binding affinity for the encoded protein, peptide or corresponding antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the encoded protein, peptide or corresponding antibody is used to form secondary immune complexes, as described above. After washing, the secondary immune complexes are contacted with a third binding ligand or antibody that has binding affinity for the second antibody, again under conditions effective and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

### B. Western Blot

It is contemplated that the methods of the current invention can include Western Blot analysis. Western Blot analysis can be used to determine the effectiveness of, for example, the up-regulation of cyclin D1 promoter activity and protein expression by β-catenin. Preferred detection methods include chemiluminescence and chromagenic detection. Standard methods for Western Blot analysis can be found in, for example, Bollag et al., 1996 or Harlow et al. 1988, herein incorporated by reference.

### C. ELISAs

As noted, it is contemplated that the ELISA may be used to study the regulation of cyclin D1 promoter activity and protein expression by β-catenin.

In one exemplary ELISA, antibodies binding to the proteins of the invention are immobilized onto a selected surface exhibiting protein affinity, such as a well in a polystyrene microtiter plate. Then, a test composition suspected of containing a marker antigen is added to the wells. After binding and washing to remove non-specifically bound immunocomplexes, the bound antigen may be detected.

Detection is generally achieved by the addition of a second antibody specific for the target protein, that is linked to a detectable label. This type of ELISA is a simple “sandwich ELISA”. Detection also may be achieved by the addition of a second antibody, followed by the addition of a third antibody that has binding affinity for the second antibody, with the third antibody being linked to a detectable label.

In another exemplary ELISA, a marker antigen is immobilized onto the well surface and then contacted with the antibodies of the invention. After binding and washing to remove non-specifically bound immunecomplexes, the bound antibody is detected. Where the initial antibodies are linked to a detectable label, the immunecomplexes may be detected directly. Again, the immunecomplexes may be detected using a second antibody that has binding affinity for the first antibody, with the second antibody being linked to a detectable label.

Another ELISA in which the proteins or peptides, are immobilized, involves the use of antibody competition in the detection. In this ELISA, labeled antibodies are added to the wells, allowed to bind to the marker protein, and detected by means of their label. The amount of marker antigen in an unknown sample is then determined by mixing the sample with the labeled antibodies before or during incubation with coated wells. The presence of marker antigen in the sample acts to reduce the amount of antibody available for binding to the well and thus reduces the ultimate signal. This is appropriate for detecting antibodies in an unknown sample, where the unlabeled antibodies bind to the antigen-coated wells and also reduces the amount of antigen available to bind the labeled antibodies.

Irrespective of the format employed, ELISAs have certain features in common, such as coating, incubating or binding, washing to remove non-specifically bound species, and detecting the bound immunecomplexes. These are described as follows:

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the control clinical or biological sample to be tested under conditions effective to allow immunecomplex (antigen/antibody) formation. Detection of the immunecomplex then requires a labeled secondary binding ligand or antibody, or a secondary binding ligand or antibody in conjunction with a labeled tertiary antibody or third binding ligand.

Under conditions effective to allow immunecomplex (antigen/antibody) formation means that the conditions preferably include diluting the antigens and antibodies with solutions such as BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 h, at temperatures preferably on the order of 25° to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immunecomplexes between the test sample and the originally bound material, and subsequent washing, the occurrence of even minute amounts of immunecomplexes may be determined.

To provide a detecting means, the second or third antibody will have an associated label to allow detection. Preferably, this will be an enzyme that will generate color development upon incubating with an appropriate chromogenic substrate. Thus, for example, one will desire to contact and incubate the first or second immunecomplex with a urease, glucose oxidase, alkaline phosphatase or hydrogen peroxidase-conjugated antibody for a period of time and under conditions that favor the development of further immunecomplex formation (e.g., incubation for 2 h at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation is then achieved by measuring the degree of color generation, e.g., using a visible spectra spectrophotometer.

In other embodiments, solution-phase competition ELISA is also contemplated. Solution phase ELISA involves attachment of a protein a related peptide to a bead, for example a magnetic bead. The bead is then incubated with sera from human and animal origin. After a suitable incubation period to allow for specific interactions to occur, the beads are washed. The specific type of antibody is the detected with an antibody indicator conjugate. The beads are washed and sorted. This complex is the read on an appropriate instrument (fluorescent, electroluminescent, spectrophotometer, depending on the conjugating moiety). The level of antibody binding can thus by quantitated and is directly related to the amount of signal present.

### D. Immunohistochemistry

The proteins and antibodies of the present invention may also be used in conjunction with both fresh-frozen and formalin-fixed, paraffin-embedded tissue blocks prepared for study by immunohistochemistry (IHC). For example, each tissue block consists of 50 mg of residual “pulverized” breast tumor tissue. The method of preparing tissue blocks from these particulate specimens has been successfully used in previous IHC studies of various prognostic factors, and is well known to those of skill in the art (Brown et al., 1990; Abbondanzo et al., 1990; Allred et al., 1990).

Briefly, frozen-sections may be prepared by rehydrating 50 ng of frozen “pulverized” breast tissue at room temperature in phosphate buffered saline (PBS) in small plastic capsules; pelleting the particles by centrifuigation; resuspending them in a viscous embedding medium (OCT); inverting the capsule and pelleting again by centrifuigation; snap-freezing in −70° C. isopentane; cutting the plastic capsule and removing the frozen cylinder of tissue; securing the tissue cylinder on a cryostat microtome chuck; and cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and embedding the block in paraffin; and cutting up to 50 serial permanent sections.

### E. FACS Analyses

Fluorescent activated cell sorting, flow cytometry or flow microfluorometry provides the means of scanning individual cells for the presence of activated or non-activated β-catenin. The method employs instrumentation that is capable of activating, and detecting the excitation emissions of labeled cells in a liquid medium. FACS is unique in its ability to provide a rapid, reliable, quantitative, and multiparameter analysis on either living or fixed cells. The antibodies of the present invention provide a useful tool for the analysis and quantitation of markers of individual cells.

### F. Gel-shift Assay

The gel shift assay or electrophoretic mobility shift assay (EMSA) is used to detect the interactions between DNA binding proteins and their cognate DNA recognition sequences, in both a qualitative and quantitative manner. The technique was originally developed for DNA binding proteins, but has since been extended to allow detection of RNA binding proteins due to their interaction with a particular RNA sequence.

In a general gel-shift assay, purified proteins or crude cell extracts are incubated with a 32P-radiolabeled DNA or RNA probe, followed by separation of the complexes from the free probe through a nondenaturing polyacrylamide gel. The complexes will migrate more slowly through the gel than unbound probe. Depending on the activity of the binding protein, a radiolabeled probe may be either double-stranded or single-stranded. For the detection of DNA binding proteins such as transcription factors, either purified or partially purified proteins, or nuclear cell extracts are used. For detection of RNA binding proteins, either purified or partially purified proteins, or nuclear or cytoplasmic cell extracts are used. The specificity of the DNA or RNA binding protein for the putative binding site is established by competition experiments using DNA or RNA fragments or oligonucleotides containing a binding site for the protein of interest, or other unrelated sequence. The differences in the nature and intensity of the complex formed in the presence of specific and nonspecific competitor allows identification of specific interactions. (http://www.shpromega.com.cn/gelshfaq.html#q01)

### G. In vivo Imaging

The invention also provides in vivo methods of imaging β-catenin activation using antibody conjugates. The term “in vivo imaging” refers to any non-invasive method that permits the detection of a labeled antibody, or fragment thereof, that specifically binds to cancer cells located in the body of an animal or human subject.

The imaging methods generally involve administering to an animal or subject an imaging-effective amount of a detectably-labeled βcatenin, cyclin D1 or α-actin specific antibody or fragment thereof (in a pharmaceutically effective carrier), such as an antibody to β-catenin, cyclin D1 or α-actin, and then detecting the location of the labeled antibody in the sample cell. The detectable label is preferably a spin-labeled molecule or a radioactive isotope that is detectable by non-invasive methods.

An “imaging effective amount” is an amount of a detectably-labeled antibody, or fragment thereof, that when administered is sufficient to enable later detection of binding of the antibody or fragment to cancer tissue. The effective amount of the antibody-marker conjugate is allowed sufficient time to come into contact with reactive antigens that be present within the tissues of the patient, and the patient is then exposed to a detection device to identify the detectable marker.

Antibody conjugates or constructs for imaging thus have the ability to provide an image of the tumor, for example, through magnetic resonance imaging, x-ray imaging, computerized emission tomography and the like. Elements particularly useful in Magnetic Resonance Imaging (“MRI”) include the nuclear magnetic spin-resonance isotopes ¹⁵⁷Gd, ⁵⁵Mn, ¹⁶²Dy, ⁵²Cr, and ⁵⁶Fe, with gadolinium often being preferred. Radioactive substances, such as technicium^{99m} or indium¹¹¹, that may be detected using a gamma scintillation camera or detector, also may be used. Further examples of metallic ions suitable for use in this invention are 123I, ¹³¹I, 131I, ⁹⁷Ru, ⁶⁷Cu, ⁶⁷Ga, 125I, ⁶⁸Ga, ⁷²As, ⁸⁹Zr, and ²⁰¹T1.

A factor to consider in selecting a radionuclide for in vivo diagnosis is that the half-life of a nuclide be long enough so that it is still detectable at the time of maximum uptake by the target, but short enough so that deleterious radiation upon the host, as well as background, is minimized. Ideally, a radionuclide used for in vivo imaging will lack a particulate emission, but produce a large number of photons in a 140-2000 keV range, which may be readily detected by conventional gamma cameras.

A radionuclide may be bound to an antibody either directly or indirectly by using an intermediary functional group. Intermediary functional groups which are often used to bind radioisotopes which exist as metallic ions to antibody are diethylenetriaminepentaacetic acid (DTPA) and ethylene diaminetetracetic acid (EDTA).

Administration of the labeled antibody may be local or systemic and accomplished intravenously, intra-arterially, via the spinal fluid or the like. Administration also may be intradermal or intracavitary, depending upon the body site under examination. After a sufficient time has lapsed for the labeled antibody or fragment to bind to the diseased tissue, in this case cancer tissue, for example 30 min to 48 h, the area of the subject under investigation is then examined by the imaging technique. MRI, SPECT, planar scintillation imaging and other emerging imaging techniques may all be used.

The distribution of the bound radioactive isotope and its increase or decrease with time is monitored and recorded. By comparing the results with data obtained from studies of clinically normal individuals, the presence and extent of the diseased tissue can be determined.

The exact imaging protocol will necessarily vary depending upon factors specific to the patient, and depending upon the body site under examination, method of administration, type of label used and the like. The determination of specific procedures is, however, routine to the skilled artisan. Although dosages for imaging embodiments are dependent upon the age and weight of patient, a one time dose of about 0.1 to about 20 mg, more preferably, about 1.0 to about 2.0 mg of antibody-conjugate per patient is contemplated to be useful.

### VIII. MODULATOR SCREENING

The present invention also provides methods of screening for modulators, e.g., activators, inhibitors, enhancers, etc., of β-catenin. Such modulators would be useful to alter β-catenin activity in a patient, for the treatment of a number of cancers. Thus, the invention provides assays for β-catenin modulation, where the subcellular localization of β-catenin at a cytoplasm, nucleus or plasma membrane determines activation of the protein. The expression of cyclin D1 can also be used as a reporter for β-catenin activity.

“Inhibitors,” “activators,” and “modulators” of β-catenin refer to any inhibitory or activating molecules identified using in vitro and in vivo assays for β-catenin, e.g., agonists, antagonists, and their homologs and mimetics. Inhibitors are compounds that decrease, block, prevent, delay activation, inactivate, desensitize, or down regulate β-catenin, e.g., antagonists. Activators are compounds that increase, open, activate, facilitate, enhance activation, sensitize or up-regulate β-catenin, e.g., agonists. Modulators include genetically-modified versions of β-catenin, e.g., with altered activity, as well as naturally-occurring and synthetic ligands, antagonists, agonists, small chemical molecules and the like. Such assays for inhibitors and activators include, e.g., expressing β-catenin protein in cells or cell membranes, applying putative modulator compounds, and then determining the functional effects on β-catenin, as described above. Samples or assays comprising β-catenin that are treated with a potential activator, inhibitor, or modulator are compared to control samples without the inhibitor, activator, or modulator to examine the extent of inhibition. Control samples (untreated with inhibitors) are assigned a relative β-catenin activity value of 100%. Inhibition of β-catenin, or blocking the pathway to form β-catenin is achieved when the β-catenin activity value relative to the control is about 80%, preferably 50%, more preferably 25-1%. Activation of β-catenins is achieved when the β-catenin activity value relative to the control is 110%, more preferably 150%, more preferably 200-500%, more preferably 1000-3000% higher.

“Biologically active” β-catenin refers to a β-catenin protein, or a nucleic acid encoding the β-catenin protein, having activity as a cell adhesion protein.

In numerous embodiments of this invention, assays will be performed to detect compounds that affect β-catenin activity in a cell or cell membrane. Such assays can involve the identification of compounds that interact with β-catenin proteins, either physically or genetically, and can thus rely on any of a number of standard methods to detect physical or genetic interactions between compounds. Such assays can also involve the detection of β-catenin in a cell or cell membrane, either in vitro or in vivo, and can thus involve the detection of transduction activity in the cell through any standard assay, e.g., by measuring ion flux, changes in membrane potential, and the like. Such cell-based assays can be performed in any type of cell, e.g., a breast cell that naturally expresses β-catenin, or a cultured cell that produces β-catenin due to recombinant expression.

In any of the binding or functional assays described herein, in vivo or in vitro, β-catenin, or any derivative, variation, homolog, or fragment of β-catenin, can be used. In numerous embodiments, a fragment of β-catenin can be used. Similarly, cyclin D1, or any derivative, variation, homolog, or fragment of cyclin D1 can be used.

In certain embodiments, assays will be performed to identify molecules that physically or genetically interact with β-catenin. Such molecules can be any type of molecule, including polypeptides, polynucleotides, amino acids, nucleotides, carbohydrates, lipids, or any other organic or inorganic molecule. Such molecules may represent molecules that normally interact with β-catenin to effect cell adhesion or may be synthetic or other molecules that are capable of interacting with β-catenin and which can potentially be used to modulate β-catenin activity in cells, or used as lead compounds to identify classes of molecules that can interact with and/or modulate β-catenin. Such assays may represent physical binding assays, such as affinity chromatography, immunoprecipitation, two-hybrid screens, or other binding assays, or may represent genetic assays as described infra.

### IX. SAMPLE PREPARATION

A sample is needed in order to perform the method of the current invention. A sample is defined herein as a cell, tissue, blood sample, cellular extract, biological fluid, serum/plasma, or a biopsy sample. This sample can be obtain in a variety of ways that are known in the art, including a fine needle aspiration, core needle biopsy, surgical biopsy, vacuum-assisted biopsy, a biopsy using an advanced breast biopsy instrument, a surgical specimen, a paraffin embedded tissue or a frozen tissue imprint. Samples can be obtained by the specific biopsy as described herein.

### A. Fine Needle Aspiration Biopsy (FNAB)

FNAB uses a thin needle which can be guided into the area of the breast abnormality or tumor while the doctor is palpating the lump. Ultrasound or stereotactic needle biopsy may also be used to guide the needle. With ultrasound, the needle can be observed on a screen as it moves toward and into the mass. For stereotactic needle biopsy, computers map the exact location of the mass using mammograms taken from two angles. Then a computer guides the needle to the right spot.

### B. Core Needle Biopsy

The needle used in core biopsies is larger than that used in FNAB. A small cylinder of tissue about 1.5 to 3 mm in diameter and 12 mm long is removed from the breast tumor. The biopsy is done under local anesthesia. As with FNAB, a core biopsy can sample tumors felt by the doctor as well as smaller ones pinpointed by ultrasound or stereotactic methods.

### C. Surgical Biopsy

In some cases, surgery may be needed to remove all or part of the lump for microscopic examination. An excisional biopsy is used to remove an entire lesion (breast abnormality such as a mass or area containing calcifications) as well as a surrounding margin of normal-appearing breast tissue. This biopsy is commonly done under a local anesthesia.

A wire localization may be used during an excisional breast biopsy for small or hard to locate tumors. A thin hollow needle is placed into the breast and x-ray pictures are used to guide the needle. A thin wire is inserted through the center of the needle. A small hook at the end of the wire keeps it in place. The hollow needle is then removed, and the surgeon uses the wire as a guide to locate the abnormal area to be removed.

### D. Other Stereotactic Biopsies

During the past few years, two new devices have been invented that can be guided by stereotactic methods and can remove more tissue than a core biopsy. The Mammotome, also known as vacuum-assisted biopsy, uses suction to draw tissue into an opening in the side of a cylinder inserted into the breast tissue. A rotating knife then cuts the tissue samples from the rest of the breast. This method usually removes about twice as much tissue as core biopsies. The ABBI method (short for Advanced Breast Biopsy Instrument) uses a rotating circular knife to remove a large cylinder of tissue. Both the Mammotome and ABBI instruments have been recently approved by the US Food and Drug Administration (FDA) for use in diagnosis of breast abnormalities. But, breast specialists still disagree about when each of these instruments should be used for diagnosis of non-palpable abnormalities, and whether some should be used at all. There is even more disagreement about whether the ABBI should be used in some situations to replace an excisional biopsy with wire localization as a lumpectomy procedure.

### X. CANCER TREATMENT

Compositions having an effective amount of a compound that modulates β-catenin activity for therapeutic administration to a person with cancer, optionally in combination with an effective amount of a second agent, for example a chemotherapeutic agent or any other anti-cancer agent are contemplated. These modulators include genetically-modified versions of β-catenin, e.g., with altered activity, as well as naturally-occurring and synthetic ligands, antagonists, agonists, small chemical molecules and the like.

### A. Pharmaceutical Compositions

The modulator compositions will generally be dissolved or dispersed in a pharmaceutically acceptable carrier or aqueous medium. The phrases “pharmaceutically or pharmacologically acceptable” refer to molecular entities and compositions that do not produce an adverse, allergic or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredients, its use in the therapeutic compositions is contemplated. Supplementary active ingredients, such as other anti-cancer agents, can also be incorporated into the compositions.

### B. Routes of Administration

Compounds may be formulated for parenteral administration as well for as other administration methods such as intravenous, intramuscular or intratumoral injection, other pharmaceutically acceptable forms include, e.g., tablets or other solids for oral administration; time release capsules; and any other form currently used, including cremes, lotions, rinses, inhalants and the like.

The expression vectors and delivery vehicles of the present invention may include classic pharmaceutical preparations. Administration of these compositions according to the present invention will be via any common route so long as the target tissue is available via that route. This includes oral, nasal or topical. Alternatively, administration may be by, e.g., orthotopic, intradermal, subcutaneous, intramuscular, intraperitoneal or intravenous injection. Such compositions would normally be administered as pharmaceutically acceptable compositions, described supra.

The vectors of the present invention are advantageously administered in the form of injectable compositions either as liquid solutions or suspensions. Solid forms suitable for solution in, or suspension in, liquid prior to injection also may be prepared. These preparations also may be emulsified. A typical composition for such purposes comprises 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters, such as theyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components in the pharmaceutical are adjusted according to well known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions can take the form of solutions, suspensions, tablets, pills, capsules, sustained release formulations or powders. When the route is topical, the form may be a cream, ointment, salve or spray.

An effective amount of the therapeutic agent is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual.

### C. Treatment Protocols

The treatment of human cancers using a modulator of β-catenin activity is contemplated in the current invention. For gene modulators, this may be achieved by introduction of the desired modulator gene through the use of a viral or non-viral vector to carry the β-catenin sequences to efficiently infect the tumor, or pre-tumorous tissue. Viral vectors will preferably be an adenoviral, a retroviral, a vaccinia viral vector or adeno-associated virus as described hereinabove uro-cacho et al., 1992). These vectors are preferred because they have been successfully used to deliver desired sequences to cells and tend to have a high infection efficiency.

β-catenin modulators may be administered parenterally or orally in dosage unit formulations containing standard, well known non-toxic physiologically acceptable carriers, adjuvants, and vehicles as desired. The term parenteral as used herein includes subcutaneous injections, intravenous, intramuscular, intra-arterial injection, or infusion techniques. A β-catenin modulator, may be delivered to the patient before, after or concurrently with the other anti-cancer agents. A typical treatment course may, for example, comprise about six doses delivered over a 7 to 21 day period. Upon election by the clinician the regimen may be continued six doses every three weeks or on a less frequent (monthly, bimonthly, quarterly etc.) basis. Of course, these are only exemplary times for treatment, and the skilled practitioner will readily recognize that many other time-courses are possible.

Regional delivery of a β-catenin modulator, will be an efficient method for delivering a therapeutically effective dose to counteract the clinical disease. Likewise, the chemotherapy may be directed to a particular effected region. Alternatively systemic delivery of either, or both, agent may be appropriate. The therapeutic composition of the present invention is administered to the patient directly at the site of the tumor. This is in essence a topical treatment of the surface of the cancer. The volume of the composition should usually be sufficient to ensure that the entire surface of the tumor is contacted by a β-catenin modulator, and second agent. In one embodiment, administration simply entails injection of the therapeutic composition into the tumor. In another embodiment, a catheter is inserted into the site of the tumor and the cavity may be continuously perfused for a desired period of time.

A major challenge in clinical oncology is that many tumor cells are resistant to chemotherapeutic treatment. One goal of the inventors'efforts has been to find ways to improve the efficacy of chemotherapy. In the context of the present invention, a β-catenin modulator, can be combined with any of a number of conventional chemotherapeutic regimens. Patients to be treated with a β-catenin modulator may, but need not, have received previous surgical, chemo- radio- or gene therapeutic treatments.

Clinical responses may be defined by acceptable measure. For example, a complete response may be defined by the disappearance of all measurable disease for at least a month. Whereas a partial response may be defined by a 50% or greater reduction of the sum of the products of perpendicular diameters of all evaluable tumor nodules or at least 1 month with no tumor sites showing enlargement. Similarly, a mixed response may be defined by a reduction of the product of perpendicular diameters of all measurable lesions by 50% or greater with progression in one or more sites.

Of course, the above-described treatment regimes may be altered in accordance with the knowledge gained from clinical trials such as those described herein. Those of skill in the art will be able to take the information disclosed in this specification and optimize treatment regimes based on the clinical trials described in the specification.

### D. Clinical Trials

Human treatment protocols may be developed using β-catenin modulation, alone or in combination with other anti-cancer drugs. The β-catenin modulation, and anti-cancer drug treatment will be of use in the clinical treatment of various cancers such as breast cancer. Such treatment will be particularly useful tools in anti-tumor therapy, for example, in treating patients with breast cancers that are resistant to conventional chemotherapeutic regimens.

The various elements of conducting a clinical trial, including patient treatment and monitoring, will be known to those of skill in the art in light of the present disclosure. The following information is being presented as a general guideline for use in establishing the β-catenin modulation, in clinical trials.

Patients with advanced, metastatic breast, epithelial ovarian carcinoma, pancreatic, colon, or other cancers chosen for clinical study will typically have failed to respond to at least one course of conventional therapy.

In regard to the β-catenin modulation therapy, a β-catenin modulator, may be administered alone or in combination with the other anti-cancer drug. The administration may be directly into the tumor, or in a systemic manner. The starting dose may be anywhere from 0.01 to 5.0 mg/kg body weight. Three patients may be treated at each dose level in the absence of grade>3 toxicity. Dose escalation may be done by 100% increments (e.g. 0.5 mg, 1 mg, 2 mg, 4 mg) until drug related grade 2 toxicity is detected. Thereafter dose escalation may proceed by 25% increments. The administered dose may be fractionated equally into multiple infusions, separated by 1 to 12 hours if the lot of anti-cancer drug exceed 5 EU/kg for any given patient.

The β-catenin modulator, and/or the other anti-cancer drug combination, may be administered over a short infusion time or at a steady rate of infusion over a 1 to 356 day period. The β-catenin modulator, infusion may be administered alone or in combination with an anti-cancer drug or surgery. The infusion given at any dose level will be dependent upon the toxicity achieved after each. Hence, if Grade II toxicity was reached after any single infusion, or at a particular period of time for a steady rate infusion, further doses should be withheld or the steady rate infusion stopped unless toxicity improved. Increasing doses of the β-catenin modulator, in combination with an anti-cancer drug will be administered to groups of patients until approximately 60% of patients show unacceptable Grade III or IV toxicity in any category. Doses that are ⅔ of this value could be defined as the safe dose.

Physical examination, tumor measurements, and laboratory tests should, of course, be performed before treatment and at intervals of about 3-4 weeks later. Laboratory studies can include mammograms, CBC, differential and platelet count, urinalysis, SMA-12-100 (liver and renal function tests), coagulation profile, and any other appropriate chemistry studies to determine the extent of disease, or determine the cause of existing symptoms. Also appropriate biological markers in serum could be monitored.

To monitor disease course and evaluate the anti-tumor responses, it is contemplated that the patients should be examined for appropriate tumor markers every 2-6 weeks, if initially abnormal. Clinical responses may be defined by acceptable measure. For example, a complete response may be defined by the disappearance of all measurable disease for at least a month. Whereas a partial response may be defined by a 50% or greater reduction of the sum of the products of perpendicular diameters of all evaluable tumor nodules or at least 1 month with no tumor sites showing enlargement. Similarly, a mixed response may be defined by a reduction of the product of perpendicular diameters of all measurable lesions by 50% or greater with progression in one or more sites.

### X. COMBINATION THERAPIES

In order to increase the effectiveness of the therapy by β-catenin modulation as described in the present invention, it may be desirable to combine these compositions with yet other agents effective in the treatment of a cancer such as breast cancer.

In the context of the present invention, it is therefore contemplated that the β-catenin modulation therapy will be used in combination with other anticancer-therapies known in the art for treating cancers that have an increased β-catenin activation. A variety of cancers including pre-cancers, tumors, malignant cancers can be treated according to the methods of the present invention. Some of the cancer types contemplated for treatment in the present invention include breast,

prostate, liver, myelomas, bladder, blood, bone, bone marrow, brain, colon, esophagus, gastrointestine, head, kidney, lung, nasopharynx, neck, ovary, skin, stomach, and uterus cancers. The treatment of breast cancer is preferred.

The administration of the other anti-cancer therapy or surgical procedure may precede or follow the β-catenin modulation therapy by intervals ranging from minutes to days to weeks. In embodiments where the other anti-cancer therapy and the β-catenin modulation therapy are administered together, one would generally ensure that a significant period of time did not expire between the time of each delivery. In such instances, it is contemplated that one would administer to a patient both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the other anti-cancer therapy and the modulation of β-catenin activity will be required in the preferential canter treatment regime.. Various combinations may be employed, where the other anti-cancer therapy agent is “A” and the β-catenin modulation therapy is “B”, as exemplified below:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

Other combinations also are contemplated. The exact dosages and regimens of each agent can be suitable altered by those of ordinary skill in the art.

Some examples of other anti-cancer therapies that may be used include chemotherapeutic agents, surgery, immunotherapy, gene therapy, hormonal therapy, or other anti-cancer therapies. It is also contemplated that other chemotherapeutics may be used, such as but not limited to, cisplatin, gemcitabine, novelbine, doxorubicin, VP16, TNF, emodin, daunorubicin, dactinomycin, mitoxantrone, procarbazine, mitomycin, carboplatin, bleomycin, etoposide, teniposide, mechlroethamine, cyclophosphamide, ifosfamide, melphalan, chlorambucil, ifosfamide, melphalan, hexamethylmelamine, thiopeta, busulfan, carmustine, lomustine, semustine, streptozocin, dacarbazine, adriamycin, 5-fluorouracil (5FU), camptothecin, actinomycin-D, hydrogen peroxide, nitrosurea, plicomycin, tamoxifen, transplatinum, vincristin, vinblastin, TRAIL, or methotrexate.

### XI. KITS

The materials and reagents required for detecting β-catenin activation in a sample may be assembled together in a kit. In one embodiment, such a kit generally will comprise anti-β-catenin, cyclin D1 or α-actin antibodies; and reagents to detect the formation of an antigen-antibody complex. In one embodiment, the anti-anti-β-catenin, cyclin D1 or α-actin antibodies may be antibodies to mutated versions of the β-catenin, cyclin D1 or α-actin protein.

The materials and reagents required for determining an individuals survival rate for certain cancer types, such as breast cancers, based on the activation of β-catenin, may also be assembled together in a kit. Such a kit will generally comprise reagents for transfecting cyclin D1 and β-catenin and optionally a fluorescent probe; and reagents to detect the binding of these probes to the proteins or antibodies. The fluorophore may comprise: Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine, and Texas Red.

In each case, the kits will preferably comprise distinct containers for each individual reagent; antibody type; and probe. Each biological agent will generally be suitable aliquoted in their respective containers. The container means of the kits will generally include at least one vial or test tube. Flasks, bottles and other container means into which the reagents are placed and aliquoted are also possible. The individual containers of the kit will preferably be maintained in close confinement for commercial sale. Suitable larger containers may include injection or blow-molded plastic containers into which the desired vials are retained. Instructions may be provided with the kit.

In further embodiments, the invention provides immunological kits for use in detecting β-catenin activation, e.g., in biological samples. Such kits will generally comprise one or more antibodies that have immunospecificity for the β-catenin, cyclin D1 or α-actin proteins or peptides. Thus, such antibodies can be used to identify β-catenin activation in cancer cells by using immunohistochemical methods such as immunohistochemical staining or ELISA's.

As the β-catenin, cyclin D1 or α-actin proteins or peptide antibodies may be employed to β-catenin activation either or both of such components may be provided in the kit. The immunodetection kits will thus comprise, in suitable container means, a β-catenin, cyclin D1 or α-actin polypeptide/protein, a first antibody that binds to such a protein or peptide, and an immunodetection reagent.

In certain embodiments, the β-catenin, cyclin D1 or α-actin proteins or peptides, or the first antibody that binds to the protein or peptide, such as an anti-β-catenin, cyclin D1 or α-actin antibody as described above, may be bound to a solid support, such as a column matrix or well of a microtitre plate.

The immunodetection reagents of the kit may take any one of a variety of forms, including those detectable labels that are associated with, or linked to, the given antibody or antigen itself. Detectable labels that are associated with or attached to a secondary binding ligand are also contemplated. Exemplary secondary ligands are those secondary antibodies that have binding affinity for the first antibody or antigen.

Further suitable immunodetection reagents for use in the present kits include the two-component reagent that comprises a secondary antibody that has binding affinity for the first antibody or antigen, along with a third antibody that has binding affinity for the second antibody, wherein the third antibody is linked to a detectable label.

As noted above in the discussion of antibody conjugates, a number of exemplary labels are known in the art and all such labels may be employed in connection with the present invention. Radiolabels, nuclear magnetic spin-resonance isotopes, fluorescent labels and enzyme tags capable of generating a colored product upon contact with an appropriate substrate are suitable examples.

The kits may contain antibody-label conjugates either in fully conjugated form, in the form of intermediates, or as separate moieties to be conjugated by the user of the kit. The kits may further comprise a suitably aliquoted composition of a β-catenin, cyclin D1 or α-actin protein or polypeptide, whether labeled or unlabeled, as may be used to prepare a standard curve for a detection assay. The kits of the invention, regardless of type, will generally comprise one or more containers into which the biological agents are placed and, preferably, suitable aliquoted. The components of the kits may be packaged either in aqueous media or in lyophilized form.

Additional embodiments provide RT-PCR-based kits which utilize primers specific for β-catenin. It is envisioned that human tissue samples will be screened for the presence of β-catenin, cyclin D1 or α-actin transcription to determine β-catenin activation. Such samples could consist of cells, cellular fluid, needle biopsy cores, surgical resection samples, or any biological fluid. The RT-PCR-based kits will contain in suitable containers the appropriate primers, the enzymes required for reverse transcription and PCR, and other suitable buffers and reagents.

All the essential materials and reagents required for modulating β-catenin activity may be assembled together in a kit. When the components of the kit are provided in one or more liquid solutions, the liquid solution preferably is an aqueous solution, with a sterile aqueous solution being particularly preferred. For in vivo use, a chemotherapeutic agent may be formulated into a single or separate pharmaceutically acceptable syringeable composition. In this case, the container means may itself be an inhalant, syringe, pipette, eye dropper, or other such like apparatus, from which the formulation may be applied to an infected area of the body, such as the lungs or even applied to and mixed with the other components of the kit. The components of the kit may also be provided in dried or lyophilized forms. When reagents or components are provided as a dried form, reconstitution generally is by the addition of a suitable solvent. It is envisioned that the solvent also may be provided in another container means.

The kits of the present invention also will typically include a means for containing the vials in close confinement for commercial sale such as, e.g., injection or blow-molded plastic containers into which the desired vials are retained. Irrespective of the number or type of containers, the kits of the invention also may comprise, or be packaged with, an instrument for assisting with the injection/administration or placement of the ultimate complex composition within the body of an animal. Such an instrument may be an inhalant, syringe, pipette, forceps, measured spoon, eye dropper or any such medically approved delivery vehicle.

### XII. Mutagenesis

In some embodiments of the present invention, such as generating a dominant negative mutant of a particular gene or gene product, mutagenesis is performed. Where employed, mutagenesis will be accomplished by a variety of standard, mutagenic procedures. Mutation is the process whereby changes occur in the quantity or structure of an organism. Mutation can involve modification of the nucleotide sequence of a single gene, blocks of genes or whole chromosome. Changes in single genes may be the consequence of point mutations which involve the removal, addition or substitution of a single nucleotide base within a DNA sequence, or they may be the consequence of changes involving the insertion or deletion of large numbers of nucleotides.

Mutations can arise spontaneously as a result of events such as errors in the fidelity of DNA replication or the movement of transposable genetic elements (transposons) within the genome. They also are induced following exposure to chemical or physical mutagens. Such mutation-inducing agents include ionizing radiations, ultraviolet light and a diverse array of chemical such as alkylating agents and polycyclic aromatic hydrocarbons all of which are capable of interacting either directly or indirectly (generally following some metabolic biotransformations) with nucleic acids. The DNA lesions induced by such environmental agents may lead to modifications of base sequence when the affected DNA is replicated or repaired and thus to a mutation. Mutation also can be site-directed through the use of particular targeting methods.

### A. Random Mutagenesis

i) Insertional Mutagenesis

Insertional mutagenesis is based on the inactivation of a gene via insertion of a known DNA fragment. Because it involves the insertion of some type of DNA fragment, the mutations generated are generally loss-of-function, rather than gain-of-function mutations. However, there are several examples of insertions generating gain-of-function mutations (Oppenheimer et al. 1991). Insertion mutagenesis has been very successful in bacteria and Drosophila (Cooley et al. 1988) and recently has become a powerful tool in corn (Schmidt et al. 1987); Arabidopsis; (Marks et al., 1991; Koncz et al. 1990); and Antirrhinum (Sommer et al. 1990).

Transposable genetic elements are DNA sequences that can move (transpose) from one place to another in the genome of a cell. The first transposable elements to be recognized were the Activator/Dissociation elements of Zea mays (McClintock, 1957). Since then, they have been identified in a wide range of organisms, both prokaryotic and eukaryotic.

Transposable elements in the genome are characterized by being flanked by direct repeats of a short sequence of DNA that has been duplicated during transposition and is called a target site duplication. Virtually all transposable elements whatever their type, and mechanism of transposition, make such duplications at the site of their insertion. In some cases the number of bases duplicated is constant, in other cases it may vary with each transposition event. Most transposable elements have inverted repeat sequences at their termini. these terminal inverted repeats may be anything from a few bases to a few hundred bases long and in many cases they are known to be necessary for transposition.

Prokaryotic transposable elements have been most studied in E. coli and Gram negative bacteria, but also are present in Gram positive bacteria. They are generally termed insertion sequences if they are less than about 2 kB long, or transposons if they are longer. Bacteriophages such as mu and D108, which replicate by transposition, make up a third type of transposable element. elements of each type encode at least one polypeptide a transposase, required for their own transposition. Transposons often further include genes coding for function unrelated to transposition, for example, antibiotic resistance genes.

Transposons can be divided into two classes according to their structure. First, compound or composite transposons have copies of an insertion sequence element at each end, usually in an inverted orientation. These transposons require transposases encoded by one of their terminal IS elements. The second class of transposon have terminal repeats of about 30 base pairs and do not contain sequences from IS elements.

Transposition usually is either conservative or replicative, although in some cases it can be both. In replicative transposition, one copy of the transposing element remains at the donor site, and another is inserted at the target site. In conservative transposition, the transposing element is excised from one site and inserted at another.

Eukaryotic elements also can be classified according to their structure and mechanism of transportation. The primary distinction is between elements that transpose via an RNA intermediate, and elements that transpose directly from DNA to DNA.

Elements that transpose via an RNA intermediate often are referred to as retrotransposons, and their most characteristic feature is that they encode polypeptides that are believed to have reverse transcriptionase activity. There are two types of retrotransposon. Some resemble the integrated proviral DNA of a retrovirus in that they have long direct repeat sequences, long terminal repeats (LTRs), at each end. The similarity between these retrotransposons and proviruses extends to their coding capacity. They contain sequences related to the gag and pol genes of a retrovirus, suggesting that they transpose by a mechanism related to a retroviral life cycle. Retrotransposons of the second type have no terminal repeats. They also code for gag- and pol-like polypeptides and transpose by reverse transcription of RNA intermediates, but do so by a mechanism that differs from that or retrovirus-like elements. Transposition by reverse transcription is a replicative process and does not require excision of an element from a donor site.

Transposable elements are an important source of spontaneous mutations, and have influenced the ways in which genes and genomes have evolved. They can inactivate genes by inserting within them, and can cause gross chromosomal rearrangements either directly, through the activity of their transposases, or indirectly, as a result of recombination between copies of an element scattered around the genome. Transposable elements that excise often do so imprecisely and may produce alleles coding for altered gene products if the number of bases added or deleted is a multiple of three.

Transposable elements themselves may evolve in unusual ways. If they were inherited like other DNA sequences, then copies of an element in one species would be more like copies in closely related species than copies in more distant species. This is not always the case, suggesting that transposable elements are occasionally transmitted horizontally from one species to another.

ii) Chemical mutagenesis

Chemical mutagenesis offers certain advantages, such as the ability to find a full range of mutant alleles with degrees of phenotypic severity, and is facile and inexpensive to perform. The majority of chemical carcinogens produce mutations in DNA. Benzo[a]pyrene, N-acetoxy-2-acetyl aminofluorene and aflotoxin B1 cause GC to TA transversions in bacteria and mammalian cells. Benzo[a]pyrene also can produce base substitutions such as AT to TA. N-nitroso compounds produce GC to AT transitions. Alkylation of the O4 position of thymine induced by exposure to n-nitrosoureas results in TA to CG transitions.

A high correlation between mutagenicity and carcinogenity is the underlying assumption behind the Ames test (McCann et al., 1975) which speedily assays for mutants in a bacterial system, together with an added rat liver homogenate, which contains the microsomal cytochrome P450, to provide the metabolic activation of the mutagens where needed.

In vertebrates, several carcinogens have been found to produce mutation in the ras proto-oncogene. N-nitroso-N-methyl urea induces mammary, prostate and other carcinomas in rats with the majority of the tumors showing a G to A transition at the second position in codon 12 of the Ha-ras oncogene. Benzo[a]pyrene-induced skin tumors contain A to T transformation in the second codon of the Ha-ras gene.

iii) Radiation Mutagenesis

The integrity of biological molecules is degraded by the ionizing radiation. Adsorption of the incident energy leads to the formation of ions and free radicals, and breakage of some covalent bonds. Susceptibility to radiation damage appears quite variable between molecules, and between different crystalline forms of the same molecule. It depends on the total accumulated dose, and also on the dose rate (as once free radicals are present, the molecular damage they cause depends on their natural diffusion rate and thus upon real time). Damage is reduced and controlled by making the sample as cold as possible.

Ionizing radiation causes DNA damage and cell killing, generally proportional to the dose rate. Ionizing radiation has been postulated to induce multiple biological effects by direct interaction with DNA, or through the formation of free radical species leading to DNA damage (Hall, 1988). These effects include gene mutations, malignant transformation, and cell killing. Although ionizing radiation has been demonstrated to induce expression of certain DNA repair genes in some prokaryotic and lower eukaryotic cells, little is known about the effects of ionizing radiation on the regulation of mammalian gene expression (Borek, 1985). Several studies have described changes in the pattern of protein synthesis observed after irradiation of mammalian cells. For example, ionizing radiation treatment of human malignant melanoma cells is associated with induction of several unidentified proteins (Boothman et al., 1989). Synthesis of cyclin and co-regulated polypeptides is suppressed by ionizing radiation in rat REF52 cells, but not in oncogene-transformed REF52 cell lines (Lambert and Borek, 1988). Other studies have demonstrated that certain growth factors or cytokines may be involved in x-ray-induced DNA damage. In this regard, platelet-derived growth factor is released from endothelial cells after irradiation (Witte, et al., 1989).

In the present invention, the term “ionizing radiation” means radiation comprising particles or photons that have sufficient energy or can produce sufficient energy via nuclear interactions to produce ionization (gain or loss of electrons). An exemplary and preferred ionizing radiation is an x-radiation. The amount of ionizing radiation needed in a given cell generally depends upon the nature of that cell. Typically, an effective expression-inducing dose is less than a dose of ionizing radiation that causes cell damage or death directly. Means for determining an effective amount of radiation are well known in the art.

In a certain embodiments, an effective expression inducing amount is from about 2 to about 30 Gray (Gy) administered at a rate of from about 0.5 to about 2 Gy/minute. Even more preferably, an effective expression inducing amount of ionizing radiation is from about 5 to about 15 Gy. In other embodiments, doses of 2-9 Gy are used in single doses. An effective dose of ionizing radiation may be from 10 to 100 Gy, with 15 to 75 Gy being preferred, and 20 to 50 Gy being more preferred.

Any suitable means for delivering radiation to a tissue may be employed in the present invention in addition to external means. For example, radiation may be delivered by first providing a radiolabeled antibody that immunoreacts with an antigen of the tumor, followed by delivering an effective amount of the radiolabeled antibody to the tumor. In addition, radioisotopes may be used to deliver ionizing radiation to a tissue or cell.

iv) In Vitro Scanning Mutagenesis

Random mutagenesis also may be introduced using error prone PCR (Cadwell and Joyce, 1992). The rate of mutagenesis may be increased by performing PCR in multiple tubes with dilutions of templates.

One particularly useful mutagenesis technique is alanine scanning mutagenesis in which a number of residues are substituted individually with the amino acid alanine so that the effects of losing side-chain interactions can be determined, while minimizing the risk of large-scale perturbations in protein conformation (Cunningham et al., 1989).

In recent years, techniques for estimating the equilibrium constant for ligand binding using minuscule amounts of protein have been developed (Blackburn et al., 1991; U.S. Pat. Nos. 5,221,605 and 5,238,808). The ability to perform functional assays with small amounts of material can be exploited to develop highly efficient, in vitro methodologies for the saturation mutagenesis of antibodies. The inventors bypassed cloning steps by combining PCR mutagenesis with coupled in vitro transcription/translation for the high throughput generation of protein mutants. Here, the PCR products are used directly as the template for the in vitro transcription/translation of the mutant single chain antibodies. Because of the high efficiency with which all 19 amino acid substitutions can be generated and analyzed in this way, it is now possible to perform saturation mutagenesis on numerous residues of interest, a process that can be described as in vitro scanning saturation mutagenesis (Burks et al., 1997).

In vitro scanning saturation mutagenesis provides a rapid method for obtaining a large amount of structure-function information including: (i) identification of residues that modulate ligand binding specificity, (ii) a better understanding of ligand binding based on the identification of those amino acids that retain activity and those that abolish activity at a given location, (iii) an evaluation of the overall plasticity of an active site or protein subdomain, (iv) identification of amino acid substitutions that result in increased binding.

v) Random Mutagenesis by Fragmentation and Reassmbly

A method for generating libraries of displayed polypeptides is described in U.S. Pat. No. 5,380,721. The method comprises obtaining polynucleotide library members, pooling and fragmenting the polynucleotides, and reforming fragments therefrom, performing PCR amplification, thereby homologously recombining the fragments to form a shuffled pool of recombined polynucleotides.

### B. Site-Directed Mutagenesis

Structure-guided site-specific mutagenesis represents a powerful tool for the dissection and engineering of protein-ligand interactions (Wells, 1996, Braisted et al., 1996). The technique provides for the preparattion and testing of sequence variants by introducing one or more nucleotide sequence changes into a selected DNA.

Site-specific mutagenesis uses specific oligonucleotide sequences which encode the DNA sequence of the desired mutation, as well as a sufficient number of adjacent, unmodified nucleotides. In this way, a primer sequence is provided with sufficient size and complexity to form a stable duplex on both sides of the deletion junction being traversed. A primer of about 17 to 25 nucleotides in length is preferred, with about 5 to 10 residues on both sides of the junction of the sequence being altered.

The technique typically employs a bacteriophage vector that exists in both a single-stranded and double-stranded form. Vectors useful in site-directed mutagenesis include vectors such as the M13 phage. These phage vectors are commercially available and their use is generally well known to those skilled in the art. Double-stranded plasmids are also routinely employed in site-directed mutagenesis, which eliminates the step of transferring the gene of interest from a phage to a plasmid.

In general, one first obtains a single-stranded vector, or melts two strands of a double-stranded vector, which includes within its sequence a DNA sequence encoding the desired protein or genetic element. An oligonucleotide primer bearing the desired mutated sequence, synthetically prepared, is then annealed with the single-stranded DNA preparation, taking into account the degree of mismatch when selecting hybridization conditions. The hybridized product is subjected to DNA polymerizing enzymes such as E. coli polymerase I (Klenow fragment) in order to complete the synthesis of the mutation-bearing strand. Thus, a heteroduplex is formed, wherein one strand encodes the original non-mutated sequence, and the second strand bears the desired mutation. This heteroduplex vector is then used to transform appropriate host cells, such as E. coli cells, and clones are selected that include recombinant vectors bearing the mutated sequence arrangement.

Comprehensive information on the functional significance and information content of a given residue of protein can best be obtained by saturation mutagenesis in which all 19 amino acid substitutions are examined. The shortcoming of this approach is that the logistics of multiresidue saturation mutagenesis are daunting (Warren et al., 1996, Brown et al., 1996; Zeng et al., 1996; Burton and Barbas, 1994; Yelton et al., 1995; Jackson et al., 1995; Short et al., 1995; Wong et al., 1996; Hilton et al., 1996). Hundreds, and possibly even thousands, of site specific mutants must be studied. However, improved techniques make production and rapid screening of mutants much more straightforward. See also, U.S. Pat. Nos. 5,798,208 and 5,830,650, for a description of “walk-through” mutagenesis.

Other methods of site-directed mutagenesis are disclosed in U.S. Pat. Nos. 5,220,007; 5,284,760; 5,354,670; 5,366,878; 5,389,514; 5,635,377; and 5,789,166.

### XIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Cell Lines and Transfections

All cell lines were obtained from the American Type Culture

Collection and maintained in DMEM/F-12 (HyClone) with 10% (vol/vol) fetal bovine serum. Transient transfections were performed by using DC-Chol liposome provided by Leaf Huang, University of Pittsburgh. In brief, exponentially growing 293 cells and MCF7 cells were cultured in six-well plates and transfected with 0.4 μg of reporter, 0.2 μg of pCMVGa1 control, and 1 μg of effector constructs or different amounts of β-catenin expression vectors in the dose-dependent experiment or transfected with the control vector pcDNA3 (Invitrogen). The β-catenin, GSK-3 (Pap, et al., 1998), and dnTcf4 effector plasmids have been described (Korinek et al., 1997). Luciferase assays were performed 40 h after transfection and normalized through β-galactosidase activity. Each assay was performed triplicate. The β-catenin stable cell lines were generated by transfecting the 293 cells with the β-catenin phosphorylation mutant (S45Y-catenin). Individual clones were selected for resistance to 500 μg/ml G418 (Geneticin, GIBCO/BRL).

123 specimens of primary breast cancer were obtained from Shanghai Second Medical University, People's Republic of China. Their ages ranged from 26 to 87 (median 48).

### EXAMPLE 2

### Western Blot Analysis

Cell lysates were separated by SDS/PAGE and transferred onto the nitrocellulose membrane. Protein levels were determined by using antibodies that recognized myc-tagged β-catenin, cyclin D1 (purchased from NeoMarkers, Union City, Calif.), and α-actin (purchased from Oncogene Science).

### EXAMPLE 3

### Gel Mobility Shift Assays

The gel-shift assays for β-catenin/Tcf4 were performed as described (Korinek et al., 1997). Extracts were prepared from intact nuclei of different breast cancer cell lines. The probe was a double-stranded 15-nt oligomer, (CCCTTTGATCTTACC; SEQ ID NO: 2); the control oligomer was CCCTTTGGCCTTACC (SEQ ID NO: 3). The binding reaction contained 5 μg of nuclear protein, 10 ng of radiolabeled probe, and 1 μg of poly(dIdC) in 25 μl of binding buffer (60 mM KCI/1 mM EDTA/1 mM DTT/10% glycerol). Samples were incubated on ice for 30 min, and the probes were added and incubated further at room temperature for 30 min. The β-catenin/Tcf4 bands were confirmed by the competition assays with the excess of cold wild-type or control oligomers and by comparing the complexes derived from the nuclear extract of 293 cells and its β-catenin transfectants.

### EXAMPLE 4

### Immunohistochemical Staining

The immunoperoxidase staining method was modified from the avidin-biotin complex technique described previously (Hsu et al., 1981). Briefly, tissue sections were deparaffinized and dehydrated in a graded series of alcohol. They were then digested in 0055 trypsin for 15 min, blocked in 0.3% hydrogen peroxide in methanol for 15 min, and treated with 1% (v/v) normal horse serum for 30 min. The slides were incubated for 3 h at room temperature with β-catenin monoclonal antibody (1:50). After extensive washing with PBX, the slides were incubated for another 60 min at room temperature with biotinylated horse anti-mouse IgG antibody diluted 1:200 in PBS. The slides were subsequently incubated for 60 min at room temperature with avidin-biotin-peroxidase complex diluted 1:100 in PBS. The peroxidase-catalyzed product was visualized with 0.125% aminoethylcarbazole chromogen stock solution (Sigma Chemical Co.). Between steps, the slides were rinsed for 2 min in PBS (pH 7.6) three times. After light counterstaining with Mayer's modified hematoxylin (Sigma Chemical Co.), the slides were dehydrated and mounted. Negative controls, which PBS was used instead of the primary antibody, were run with each batch of staining. The prepared slides were examined by light microscopy. Those tumor cells with significantly immunostained in cytoplasm and/or nucleus considered high β-catenin activity, and those cells mainly stained at membrane were considered low β-catenin activity.

### EXAMPLE 5

### Statistical Analysis

Fisher's exact test was used to analyze for the association of β-catenin catenin activity and clinicopathological factors including TNM staging, estrogen receptor and progesterone receptor status. Survival curves were calculated by Kaplan-Meier product limit estimate. The log rank method was used to analyze the differences in survival time. The statistical analyses were performed by using SPSS software. The p value of less than 0.05 was set as the criterion for statistical significance.

### EXAMPLE 6

### Up-Regulation of Cyclin D1-Promoter Activity and the Protein Expression by β-Catenin

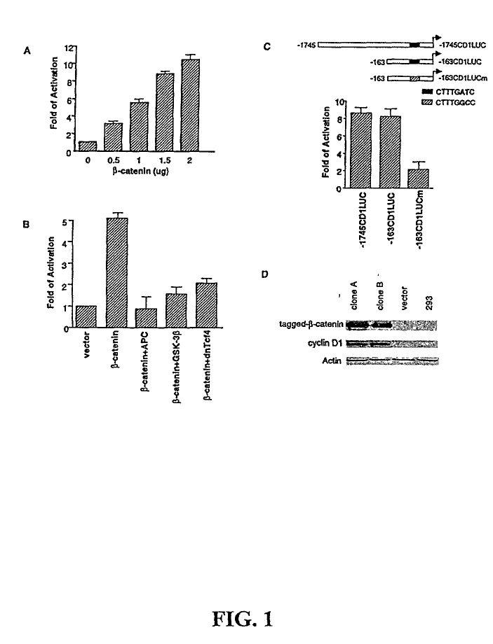

The transcriptional regulation of cyclin D1 by β-catenin was determined. The transient transfection of exogenous human β-catenin in human embryonic kidney 293 cells could activate a cyclin D1 reporter, containing 1,745 bp of the cyclin D1 promoter, up to 11-fold in a dose-dependent manner (FIG. 1A). This activation was blocked when various inhibitors of the β-catenin/Tcf4 pathway were coexpressed such as APC, GSK-3, and a dominant negative mutant of human Tcf4 (dnTcf4) (FIG. 1B) (He et al., 1998). The 293 cell line was chosen because of its low background of β-catenin activity and its previous use for studying the response to β-catenin/Tcf4-mediated transcription (Porfiri, et al., 1997).

To confirm that the Tcf4 site on cyclin D1 promoter was responsible for the activation by β-catenin, a deletion construct containing 163 bp of the cyclin D1 promoter was used as the reporter. Many known transcription factor binding sites had been eliminated from this construct, but it still contained the putative Tcf4 site (163CD1LUC). As shown in FIG. 1C, expression of β-catenin activated this reporter to a similar extent, suggesting that the responsive element remained within this deletion construct. When the Tcf4 site was mutated so that the AT was changed to GC at nucleotides 75 and 73 (163CD1LUCm), β-catenin no longer sufficiently activated the cyclin D1 gene promoter. These data indicated that the putative Tcf4 site located at 80 to 73 was responsible for the β-catenin-mediated transactivation of the cyclin D1 promoter. In addition to transient transfection, a stable cell line was generated by transfecting the 293 cells with the β-catenin phosphorylation mutant (S45Y-catenin). This mutant has been shown to resist degradation and to increase its activity to transactivate β-catenin/Tcf4-dependent transcription (Porfiri, et al., 1997). As shown in FIG. 1D, cyclin D1 protein expression in both individual stable transfectants was substantially increased (lanes 1 and 2) compared with the vector control cells (lane 3) and the parental cells (lane 4).

### EXAMPLE 7

### Correlation Between Cyclin D1 Expression and β-Catenin/Tcf4 Activity in Breast Cancer Cell Lines

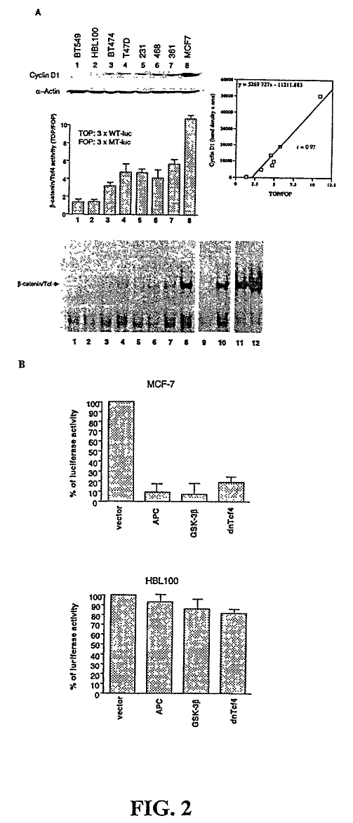

After identifying cyclin D1 as the target gene for β-catenin, the role β-catenin played in up-regulating the expression of cyclin D1 in breast cancer was studied. This possibility was tested first in breast cancer cell lines in vitro. Eight breast cancer cell lines were chosen to compare their cyclin D1 expression level and their β-catenin/Tcf4 activity. Reporter constructs that contained three repeats of wild-type (TOP) or mutant (FOP) Tcf4-binding sites (Korinek et al., 1997) were used to determine the transactivational activity of endogenous β-catenin/Tcf4. Higher ratios of these two reporter activities (TOP/FOP) indicated a higher β-catenin/Tcf4 activity. As shown in FIG. 2A (Top and Middle), cyclin D1 expression in breast cancer cells highly correlated with the β-catenin/Tcf4 activity. The eight cell lines tested could be roughly divided into three groups. BT549 and HBL100 cell lines, which expressed almost no detectable cyclin D1, had the background transactivating activity of β-catenin/Tcf4 (TOP/FOP=1). In contrast, MCF-7, which expressed the highest level of cyclin D1 protein, had the most significant β-catenin/Tcf4 activity (TOP/FOP=10). In the other five cell lines, cyclin D1 expression was consistently moderate, as were β-catenin/Tcf4 activities. By linear regression, cyclin D1 expression was demonstrated as being proportionally correlated with β-catenin/Tcf4 activity (r=0.97). In addition to the reporter assay, the β-catenin/Tcf4 activity was also confirmed by gel-shift assay. Consistent with reporter activity and cyclin D1 expression levels, β-catenin/Tcf4-binding activity was not detectable for either BT549 or BBL100 cells and was detected most strongly in MCF-7 cells as shown in FIG. 2A Bottom.

To further address whether cyclin D1 promoter activity is indeed regulated by β-catenin in these breast cancer cell lines, the cyclin D1 reporter was co-transfected with different negative regulators of the β-catenin/Tcf4 pathway in MCF-7 cells. As shown in FIG. 2B Top, the reporter activity of cyclin D1 promoter was significantly reduced. This reduction of activity could be reversed when β-catenin was co-expressed. In contrast, cyclin D1 reporter activity was not affected by the expression of APC, GSK-3, or dnTcf4 in HBL100 cells in which both β-catenin activity and cyclin D1 expression were low (FIG. 2B Bottom). Our data, therefore, support a substantial role for β-catenin in activating cyclin D1 expression in breast cancer cells.

### EXAMPLE 8

### Correlation Between Activated β-Catenin and Cyclin D1 Overexpression and Their Association with Poor Patient Survival Rate

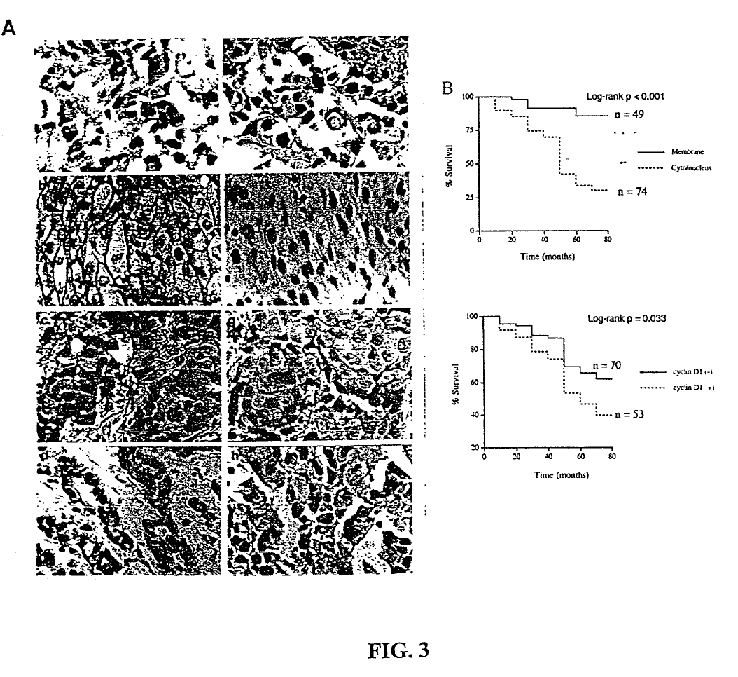

Because cyclin D1 overexpression has been well-documented in patients with breast cancer, the clinical verification of whether β-catenin activity truly contributed to the cyclin D1 overexpression in breast cancer tissues was sought. Both cyclin D1 expression and β-catenin activity were determined in 123 primary human breast cancer tissues (age: 26-87 years old; medium: 48 years old) by immunohistochemical staining (FIG. 3A). β-catenin activity was determined by its subcellular localization (Rimm, et al., 1999; Fukuchi et al., 1998). It has been well documented that accumulated β-catenin in cytoplasm and/or the nucleus increased when cells had stabilized β-catenin and, consequently, the activated β-catenin/Tcf4 activity. In contrast, β-catenin was localized solely at the plasma membrane of cells when its transactivation activity was low. The correlation between the β-catenin localization and its transactivation activity were also confirmed in various breast cell lines listed in FIG. 2.

As shown in Table 1, the subcellular localization of β-catenin and cyclin D1 was significantly correlated based on the analysis by Spearman rank correlation (r=0.6, P<0.001). The samples stained as either high β-catenin activity with high cyclin D1 expression (40%) or low β-catenin activity with negative cyclin D1 staining (37%). It is worthwhile to mention that, among the 53 cases staining positive for cyclin D1, 49 cases (92%) were positive for β-catenin activity (stained in cytoplasm/nucleus). Thus, the correlation between these two molecules in the primary tumor samples was consistent with in vitro data from the breast cancer cell lines (FIG. 2A). Therefore, it is believed that high β-catenin activity may significantly contribute to cyclin D1 overexpression in breast cancer. These results not only supported molecular data described above but also further strengthened their clinical biological significance.

TABLE 1
Immunoreactivity of β-catenin and cyclin D1 in
surgical specimens of breast cancer

| | Cyclin D1 Staining | | |
|---|---|---|---|
| β-Catenin Staining | Negative (n = 70) | Positive (n = 53) | Total |
| Cytoplasm/nucleus | 25 (20%) | 49 (40%) | 74 (60%) |
| Membrane Only | 45 (37%) | 4 (3%) | 49 (40%) |
| Total | 70 (57%) | 53 (43%) | 120 (100%) |

More importantly, when the prognostic significance was assessed by Kaplan-Meier analysis and log-rank test, it was found that both cyclin D1 overexpression and activated β-catenin were associated with a poorer prognosis and were negatively correlated with patient survival rates (P=0.033 and P<0.001, respectively) (FIG. 3B).

To determine whether activated β-catenin was independent of other known prognostic factors in prognosis, multivariate analyses for survival rate were also performed. Activated β-catenin was found to be a strong prognostic factor that provided additional and independent predictive information on the patient's survival rate even when other prognostic factors (lymph node metastasis, estrogen receptor and progesterone receptor status, and tumor size) were taken into account (P=0.001). Cyclin D1 overexpression was also an independent prognostic factor. However, when multivariate analysis was performed including only cyclin D1 expression and β-catenin activity, cyclin D1 was no longer an independent prognostic factor (the P values for cyclin D1 and β-catenin activity were P=0.457 and P<0.001, respectively). These results were consistent with the model that cyclin D1 overexpression could be caused by activated β-catenin in breast cancer and consequently correlated to the prognosis.

### EXAMPLE 9

### Blockage of β-catenin/Tcf4 Activity Inhibited Cell Growth

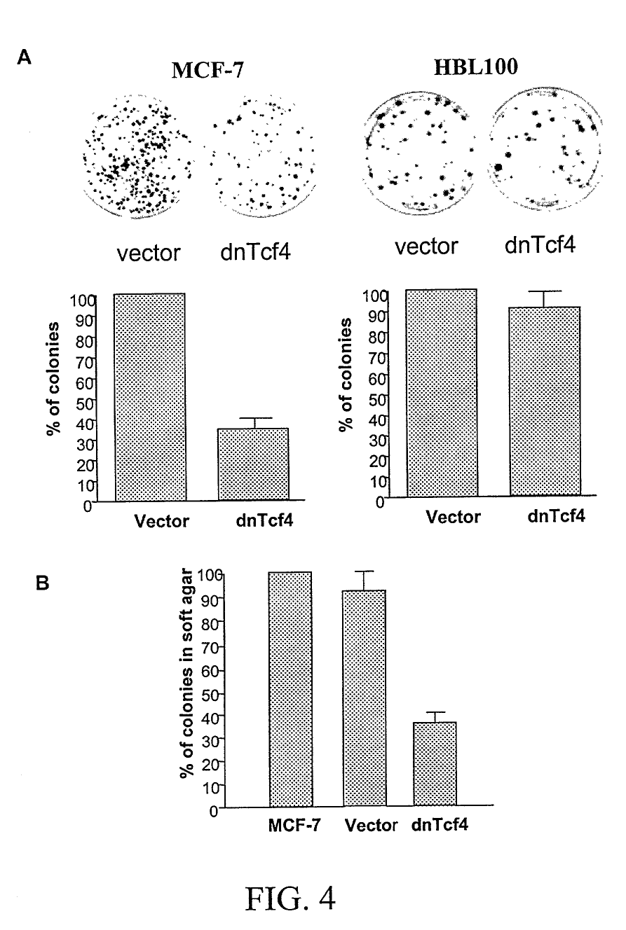

The inhibition of β-catenin using a dominant-negative Tcf4 expression construct (dnTcf4) in cells with activated β-catenin (CF-7) and in cells where β-catenin is not activated (HBL100) is shown in FIG. 4. The colony density in cells that did not express β-catenin was not altered after transfection with dnTcf4 while the colony density in MCF-7 cells was greatly reduced after transfection with dnTcf4. This shows the usefulness of the current invention in cancer therapy, demonstrating that vectors that target β-catenin can be used to block β-catenin/Tcf4 activity.

### EXAMPLE 10

### β-Catenin as a Prognostic Factor for Cancer

It is conceived that β-catenin be used as a prognostic factor for a variety of types of cancer. Examples of cancers include breast cancer, ovarian cancer, colorectal cancer, thyroid cancer, skin cancer, brain cancer, head and neck cancer, prostate, liver, myelomas, bladder, blood, bone, bone marrow, esophagus, gastrointestine, kidney, lung, nasopharynx, stomach, and uterus cancers. Thus, the present invention is directed to compositions and methods for identifying a patient at risk for the development, recurrence, or metastasis of cancer by obtaining a sample from the patient by standard means in the art, regardless of the type of cancer, and analyzing it for β-catenin activation.

In specific embodiments of the present invention, the sample, such as a sample from a breast, ovary, colon, or skin, is analyzed for β-catenin activation by transfecting a patient test sample with a cyclin D1 reporter; transfecting the sample with a β-catenin-encoding nucleic acid; and analyzing the transfectants. In other specific embodiments, the sample comprises a cell and the analysis comprises determining subcellular localization at a cytoplasm, nucleus or plasma membrane of the cell. A decrease in β-catenin activation is determined by β-catenin localization at the plasma membrane, and this is indicative of an increase in patient survival rate. An increase in β-catenin activation is determined by β-catenin accumulation at the cytoplasm or nucleus, and this is indicative of a decrease in patient survival rate.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

### Patents

U.S. Pat. No. 5,221,605

U.S. Pat. No. 5,238,808

U.S. Pat. No. 5,798,208

U.S. Pat. No. 5,830,650

U.S. Pat. No. 5,380,721

U.S. Pat. No. 5,220,007

U.S. Pat. No. 5,284,760

U.S. Pat. No. 5,354,670

U.S. Pat. No. 5,366,878

U.S. Pat. No. 5,389,514

U.S. Pat. No. 5,635,377

U.S. Pat. No. 5,789,166

### Publications

Abbondanzo et al, Breast Cancer Res. Treat., 16:182(151), 1990.

Aberle, Schwartz, Hemler, “Cadherin-catenin complex: protein interactions and their implication for cadherin function,” J. Cell Biochem., 61:514-523, 1996.

Allred et al., Breast Cancer Res. Treat., 16:182(149), 1990.

Arber, N., Hibshoosh, H., Moss, S. F., Sutter, T., Zhang, Y., Begg, M., Wang, S., Weinstein, I. B. & Holt, P. R. (1996) Gastroenterology 110, 669-674.

B'ankfalvi A, Terpe H J, Breukelmann D, Bier B, Rempe D, Pschadka G, Krech R, Lell'e R J, Boecker W, “Immunophenotypic and prognostic analysis of E-cadherin and beta-catenin expression during breast carcinogenesis and tumour progression: a comparative study with CD44.”, Histopathology (1999), 34 (1) 25-34.

Bartkova, J., Lukas, J., Muller, H., Lutzhoft, D., Strauss, M. & Bartek, J. (1994) Int. J. Cancer 11, 353-361.

Bartkova, J., Lukas, J., Strauss, M. & Bartek, J. (1994) Int. J. Cancer 58, 568-573.

Blasband, A., Schryver, B. & Papkoff, J. (1992) Oncogene 7, 153-161.

Blasband, Schryver, Papkoff, “The biochemical properties and transforming potential of human Wnt-2 are similar to Wnt-1,” Oncogene, 7:155-161, 1992.

Bohlm J, Niskanen L, Kiraly K, Kellokoski J, Eskelinen M, Hollmen S, Alhava E, Kosma VM, “Expression and prognostic value of alpha-, ss-, and gamma-catenins in differentiated thyroid carcinoma.” J Clin Endocrin Metabol, (2000) 85(12), 4806-4811.

Brown et al., Breast Cancer Res. Treat., 16:192(191), 1990.

Buckholm, I K, Nesland, J M, Karesen, R, Jacobsen, U, Borresen-Dale, A-L, (1998) J. Pathol, 185, 262-266.

Bui et al., “A novel human Wnt gene, WNT10B, maps to 12q13 and is expressed in human breast carcinomas,' Oncogene, 14:1249-1253, 1997.

Bui, T. D., Rankin, J., Smith, K., Huguet, E. L., Ruben, S., Strachan, T., Harris, A. L. & Lindsay, S. (1997) Oncogene 14, 1249-1253.

Charpin C, Bonnier P, Garcia S, Andrac L, Crebassa B, Dorel M, Lavaut M N, Allasia C, “E-cadherin and beta-catenin expression in breast medullary carcinomas.”, Int. J. Oncol. (1999), 15 (2) 285-92.

Choate and Khavari, Hum. Gene Ther., 8:895-901, 1997.

Coffin, “Retroviridae and their replication,” In: Virology, Fields et al. (eds.), New York: Raven Press, pp. 1437-1500, 1990.

Couch et al., Am. Rev. Resp. Dis., 88:394-403, 1963.

Dale et al., “Compartment switching of WNT-2 expression in human breast tumors,” Cancer Res., 56:4320-4323.

Dale, T. C., Weber-Hall, S. J., Smith, K., Huguet, E. L., Jayatilake, H., Gusterson, B. A., Shuttleworth, G., O'Hare, M. & Harris, A. L. (1996) Cancer Res. 56, 4320-4323.

Dillon D A, D'Aquila T, Reynolds A B, Fearon E R, Rimm D L, “The expression of p120ctn protein in breast cancer is independent of alpha- and beta-catenin and E-cadherin.”, Am J. Pathol. (1998), 152(1), 75-82.

Fagotto, Funayama, Gluck, Gumbinger, “Binding to cadherins antagonizes the signaling activity of β-catenin during axis formation in Xenopus, J. Cell. Biol., 132:1105-1114, 1996.

Fantl, V., Smith, R. M., Brookes, S., Dickson, C. & Peters, G. (1993) Cancer Surv. 18, 77-94.

Fenjves et al., J. Invest. Dermatol., 106:576-578, 1996.

Fitzpatrick et al., Dermatology in General Medicine, 1993.

Flotte et al., “Gene expression from adeno associated virus vector in airway epithelial cells,” Am. J. Respir. Cell Mol. Biol., 7:349-356, 1992.

Flotte, Afione, Conrad, McGrath, Solow, Oka, Zeitlin, Guggino, and Carter, “Stable in vivo expression of the cystic fibrosis transmembrane conductance regulator with an adeno-associated virus vector,” Proc Natl. Acad. Sci. USA, 90:10613-10617, 1993.

Flotte, Barraza-Ortiz, Solow, Afione, Carter, and Guggino, “An improved system for packaging recombinant adeno-associated virus vectors capable of in vivo transduction,” Gene Therapy, 2:29-37, 1995.

Fukuchi, T., Sakamoto, M., Tsuda, H., Maruyama, K., Nozawa, S. & Hirohashi, S. (1998) Cancer Res. 58, 3526-3528.

Gamallo, C, Palacios, J, Suarez, A, Pizarro, A, Quintanilla, M, Cano, A (1993) Am. J. Pathol 142, 987-993.

Gerrard et al., Nature Genet., 3:180-183, 1993.

Ghosh-Choudhury et al., “Protein IX, a minor component of the human adenovirus capsid, is essential for the packaging of full-length genomes,” EMBO J., 6:1733-1739, 1987.

Gillet, C., Fantl, V., Fisher, C., Bartek, J., Dickson, C., Barnes, D. & Peters, G. (1994) Cancer Res. 54, 1812-1817.

Graham and Prevec, “Manipulation of adenovirus vectors,” In: Gene Transfer and Expression Protocols, Murray, E. J., ed., Humana, N.J., vol. 7, 109-128, 1991.

Graham et al., “Characteristics of a human cell line transformed by DNA from human adenovirus type 5”, J. Gen. Virol., 36:59-72, 1977.

Grunhaus and Horwitz, “Adenovirus as cloning vector,” Seminar in Virology, 3:237-252, 1992.

He, T., Sparks, A. B., Rago, C., Hermeking, H., Zawel, L, da Costa, L. T., Morin, P. J., Vogelstein, B. & Kinzler, K. W. (1998) Science 281, 1509-1512.

Hermonat and Muzyczka, “Use of adeno-associated virus as a mammalian DNA cloning vector; transduction of neomycin resistance into mammalian tissue culture cells,” Proc Nat'l. Acad. Sci. USA, 81:6466-6470, 1984.

Hersdorffer et al., “Efficient gene transfer in live mice using a unique retroviral packaging line,” DNA Cell Biol., 9:713-723, 1990.

Herz and Gerard, “Adenovirus-mediated transfer of low density lipoprotein receptor gene acutely accelerates cholesterol clearance in normal mice,” Proc. Nat'l. Acad. Sci. USA 90:2812-2816, 1993.

Hsu, Raine, Fanger, “Use of avidin-biotin-peroxidase complex (ABC) in immunoperoxidase techniques: a comparison between ABC and unlabeled antibody (PAP) procedures,” J. Histochem. Cytochem., 29:577-580, 1981.

Hugh T J, Dillon S A, O'Dowd G, Getty B, Pignatelli M, Poston G J, Kinsella A R. “beta-catenin expression in primary and metastatic colorectal carcinoma.” Int J Cancer. (1999b) 12;82(4):504-11.

Hugh T J, Dillon S A, Taylor B A, Pignatelli M, Poston G J, Kinsella A R.“Cadherin-catenin expression in primary colorectal cancer: a survival analysis.” Br J Cancer. (1999a), 80(7): 1046-51.

Hunt, N C A, Douglas-Jones, A G, Jasani, B, Morgan, J M, Pignatelli, M. (1997), 430, 285-289.

Inomata “Alteration of beta-catenin expression in colonic epithelial cells of familial adenomatous polyposis patients” (1996), Cancer Res. 56, 2213-2217.

Jawhari A, Jordan S, Poole S, Browne P, Pignatelli M, Farthing M J. “Abnormal immunoreactivity of the E-cadherin-catenin complex in gastric carcinoma: relationship with patient survival.” Gastroenterology. (1997), 112(1):46-54.

Jones and Shenk, “Isolation of deletion and substitution mutants of adenovirus type 5,” Cell, 13:181-188, 1978.

Kaplitt, Leone, Samulski, Siao, Pfaff, O'Malley, and During, “Long-term gene expression and phenotypic correction suing adeno-associated virus vectors in the mammalian brain,” Nature Genetics, 8:148-154, 1994.

Karayiannakis A J, Nakopoulou L, Gakiopoulou H, Keramopoulos A, Davaris P S, Pignatelli M. “Expression patterns of beta-catenin in in situ and invasive breast cancer.” Eur J Surg Oncol. (2001), 27(1):31-36.

Karayiannakis A J, Syrigos K N, Alexiou D, Kalahanis N, Rosenberg T, Bastounis E, Pignatelli M. “Expression patterns of the novel catenin p120cas in gastrointestinal cancers.” Anticancer Res. 1999, 19(5C):4401-5.

Kasahara et al., Science, 266:1373-1376, 1994.

Korinek et al., “Constitutive transcriptional activation by a β-catenin-Tcf complex in APC-/-colon carcinoma,” Science, 275:1784-1787, 1997.

Korinek, V., Barker, N., Morin, P. J., van Wichen, D., de Weger, R., Kinzler, K. W., Vogelstein, B. & Clevers, H. (1997) Science 275, 1784-1787.

Kotin, Siniscalco, Samulski, Zhu, Hunter, McLaughlin, Muzyczka, and Bems, “Site-specific integration by adeno-associated virus,” Proc Natl Acad. Sci. USA, 87:2211-2215, 1990.

Laughlin, Cardellichio, and Coon, “Latent infection of kb cells with adeno-associated virus type 2,” J. Virol, 60:515-524, 1986.

Le Gal La Salle et al., “An adenovirus vector for gene transfer into neurons and glia in the brain,” Science, 259:988-990, 1993.

Lebkowski, McNally, Okarma, and Lerch, “Adeno-associated virus: a vector system for efficient introduction and integration of DNA into a variety of mammalian cell types,” Mol. Cell. Biol., 8:3988-3996, 1988.

Lebkowski, McNally, Okarma, and Lerch, “Adeno-associated virus: a vector system for efficient introduction and integration of DNA into a variety of mammalian cell types,” Mol. Cell. Biol., 8:3988-3996, 1988.

Lee, S W, (1996), Nature Med. 2, 776-782.

Lejeune, Huguet, Hamby, Poulsom, Harris, “Wnt5a cloning, expression, and up-regulation in human primary breast cancers,” Clin. Cancer Res., 1:215-222, 1995.

Lejeune, S., Huguet, E. L., Hamby, A., Poulsom, R. & Harris, A. L. (1995) Clin. Cancer Res. 1, 215-222.

Lu et al., J. Invest. Dermatol., 108(5):803-808, 1997.

Luo, Zhou, Cooper, Munshi, Boswell, Broxmeyer, and Srivastava, “Adeno-associated virus 2 mediated transfer and functional expression of a gene encoding the human granulocyte-macrophage colony-stimulating factor,” Blood,82 (Supp.):1,303A, 1994.

Mann et al., “Construction of a retrovirus packaging mutant and its use to produce helper-free defective retrovirus,” Cell, 33:153-159, 1983.

Markowitz et al., “A safe packaging line for gene transfer: Separating viral genes on two different plasmids,” J. Virol., 62:1120-1124, 1988.

McCarty, Christensen, and Muzyczka, “Sequences Required for Coordinate Induction of Adeno-Associated Virus p19 and p40 Promoters by Rep Protein,” J. Virol, 65:2936-2945, 1991.

McLaughlin, Collis, Hermonat, and Muzyczka, “Adeno-Associated Virus General Transduction Vectors: Analysis of Proviral Structures,” J. Virol., 62:1963-1973, 1988.

Miller, Curr. Top. Microbiol. Immunol., 158:1, 1992.

Morin, P., Sparks, A. B., Korinek, V., Barker, N., Clevers, H., Vogelstein, B. & Kinzler, K. W. (1997) Science 275, 1787-1790.

Munro, S and Pelham, H. R. (1984) EMBO J. 3, 3087-3093.

Muro-Cacho et al., “Gene transfer in human lymphocytes using a vector based on adeno-associated virus,” J Immunother. 11(4):231-7, 1992.

Muzyczka, “Use of Adeno-Associated Virus as a General Transduction Vector for Mammalian Cells,” Curr. Top. Microbiol. Immunol., 158:97-129, 1992.

Nakamura et al., In: Enzyme Immunoassays: Heterogeneous and Homogeneous Systems, Chapter 27, 1987.

Nakopoulou L, Gakiopoulou H, Keramopoulos A, Giannopoulou I, Athanassiadou P, Mavrommatis J, Davaris P S, “c-met tyrosine kinase receptor expression is associated with abnormal beta-catenin expression and favourable prognostic factors in invasive breast carcinoma.”, Histopathology, (2000), 36 (4) 313-25.

Naldini et al. (1996) Science 272:263

Nicolas and Rubinstein, “Retroviral vectors,” In: Vectors: A survey of molecular cloning vectors and their uses, Rodriguez and Denhardt (eds.), Stoneham: Butterworth, pp. 494-513, 1988.

Nusse, R., van Ooyen, A., Cox, D., Fung, Y. K. & Varmus, H. (1984) Nature (London) 307, 131-136.

Nusse, van Ooyen, Cox, Fung, Varmus, “Mode of provival activation of a utative mammary oncogene (int-1) on mouse chromosome 15,” Nature, 307:131-136, 1984.

Ohi, Dixit, Tillery, and Plonk, “Construction and replication of an adeno-associated virus expression vector that contains human .1ambda.-globin cDNA,” Gene, 89L:279-282, 1990.

Oro et al., Science, 276:817-821, 1997.

Pap, M. & Cooper, G. M. (1998) J. Biol. Chem. 273, 19929-19932.

Paskind et al., “Dependence of moloney murine leukemia virus production on cell growth,” Virology, 67:242-248, 1975.

Pennisi, Science, 281(5382):1438-9, 1441, 1998.

Polakis, “The oncogenic activation of β-catenin,” Curr. Opin. Gene Dev., 9:15-21, 1999.

Polakis, P. (1999) Curr. Opin. Gene Dev. 9, 15-21.

Polakis, P. (2000) Genes and Development 14:1837-1851.

Porfiri, E., Rubinfeld, B., Albert, I., Hovanes, K., Waterman, M. & Polakis, P. (1997) Oncogene 15, 2833-2839.

Prall, O. W. J, Rogan, E. M., Musgrove, E. A., Watts, C. K. W., Sutherland, R. L. “c-Myc or Cyclin D1 Mimics Estrogen Effects on Cyclin E-Cdk2 Activation and Cell Cycle Reentry” Mol Cell Biol, 18:8, 4499-4508, 1998.

Racher et al., Biotechnology Techniques, 9:169-174, 1995.

Renan, “Cancer genes: current status, future prospects, and applicants in radiotherapy/oncology,” Radiother. Oncol., 19:197-218, 1990.

Rich et al., “Development and analysis of recombinant adenoviruses for gene therapy of cystic fibrosis,” Hum. Gene Ther., 4:461-476, 1993.

Richmond P J, Karayiannakis A J, Nagafuichi A, Kaisary A V, Pignatelli M. “Aberrant E-cadherin and alpha-catenin expression in prostate cancer: correlation with patient survival.”, Cancer Res. (1997), 1;57(15):3189-93.

Rimm, D. L., Caca, K., Hu, G., Harrison, F. B. & Fearon, E. R. (1999) Am. J. Pathol. 154, 325-329.

Rimm, D L Sinard, J H, Morrow, J S (1995) Lab Invest. 72. 506-512.

Roelink, H., Wagenaar, E., Lopes da Silva, S. & Nusse, R. (1990) Proc. Natl. Acad. Sci. USA 87, 4519-4523.

Roelink, Wagenaar, Lopes, Da Silva, Nusse, “Wnt-3, a gene activated by proviral insertion in mouse mammary tumors, is homologous to int-1/Wnt-1 and normally expressed in mouse embryos and adult brain,” Proc. Natl. Acad. Sci., USA, 87:4519-4523, 1990.

Rosen, Sodroski, and Haseltine, “The location of cis-acting regulatory sequences in the human t-cell lymphotropic virus type III (HTLV-111/LAV) long terminal repeat,” Cell, 41:813, 1988.

Rubinfeld et al., “Stabilization of β-catenin by genetic defects in melanoma cell lines,” Science, 275:1790-1792, 1997.

Rubinfeld, B., Robbins, P., El-Gamil, M., Albert, I., Porfiri, E. & Polakis, P. (1997) Science 275, 1790-1792.

Samulski, Chang, and Shenk, “Helper-free stocks of recombinant adeno-associated viruses: Normal integration does not require viral gene expression,” J. Virol, 63:3822-3828, 1989.

Samulski, Zhu, Xiao, Brook, Housman, Epstein, and Hunter, “Targeted integration of adeno-associated virus (AAV) into human chromosome 19,” EMBO J, 10:3941-3950, 1991.

Sanson, White, Vincent, “Uncoupling cadherin-based adhesion from wingless signaling in Drosophila,” Nature, 383:627-630, 1996.

Setoguchi et al., J. Invet. Dermatol., 102:415-421, 1994.

Shelling and Smith, “Targeted integration of transfected and infected adeno-associated virus vectors containing the neomycin resistance gene,” Gene Therapy, 1:165-169, 1994.

Shimoyama, Y, Nagafuchi, A, Fujita, S, Gotoh, M et al., Cancer Res. (1992), 52, 5770-5774.

Shtutman, M., Zhurinsky, J., Simcha, I., Albanese, C., D'Amico, M., Pestell, R. & Ben-Ze'ev, A. (1999) Proc. Natl. Acad. Sci. USA 96, 5522-5527.

Silye R, Karayiannakis A J, Syrigos K N, Poole S, van Noorden S, Batchelor W, Regele H, Sega W, Boesmueller H, Krausz T, Pignatelli M. “E-cadherin/catenin complex in benign and malignant melanocytic lesions.” J Pathol. (1998), 186(4):350-5.

Sitonen, S M, Kononen, J T, Helin, H J, Rantala, I S, Holli, K A, Isola, J J (1996) Am. J. Clin. Pathol, 105, 394-402.

Stratford-Perricaudet and Perricaudet, In: Human Gene Transfer, O. Cohen-Haguenauer et al., eds., John Libbey Eurotext, France, 51-61, 1991.

Stratford-Perricaudet et al, Hum. Gene. Ther., 1:241-256, 1990.

Syrigos K N, Harrington K, Waxman J, Krausz T, Pignatelli M. “Altered gamma-catenin expression correlates with poor survival in patients with bladder cancer.” J Urol. (1998b), 160(5):1889-93

Syrigos K N, Karayiannakis A, Syrigou E I, Harrington K, Pignatelli M. “Abnormal expression of p120 correlates with poor survival in patients with bladder cancer.” Eur J Cancer. (1998a), 34(13):2037-40.

Temin, “Retrovirus vectors for gene transfer: Efficient integration into and expression of exogenous DNA in vertebrate cell genome,” In: Gene Transfer, Kucherlapati (ed.), New York: Plenum Press, pp. 149-188, 1986.

Tetsu, O. & McCormick, F. (1999) Nature (London) 398, 422-426.

Top et al., “Immunization with live types 7 and 4 adenovirus vaccines. II. Antibody response and protective effect against acute respiratory disease due to adenovirus type 7,” J. Infect. Dis., 124:155-160, 1971.

Tratschin, Miller, Smith, and Carter, “Adeno-associated virus vector for high-frequency integration, expression and rescue of genes in mammalian cells,” Mol. Cell. Biol., 5:3258-3260, 1985.

Tratschin, Miller, Smith, and Carter, “Adeno-associated virus vector for high-frequency integration, expression and rescue of genes in mammalian cells,” Mol. Cell. Biol., 5:3258-3260, 1985.

Tratschin, West, Sandbank, and Carter, “A human parvovirus, adeno-associated virus, as a eucaryotic vector: transient expression and encapsidation of the procaryotic gene for chloramphenicol acetyltransferase,” Mol. Cell. Biol., 4:2072-2081, 1984.

Tsukamoto, A. S., Grosschedl, R., Guzman, R. C., Parslow, T. & Varmus, H. E (1988) Cell 55, 619-625.

Tsukamoto, Grosschedl, Guzman, Parslow, Varmus, “Expresison of the int-1 gene in transgenic mice is associated with mammary gland hyperplasia and adenocarcinomas in male and female mice,” Cell, 55:619-625, 1988.

Walsh, Nienhuis, Samulski, Brown, Miller, Young, and Liu, “Phenotypic correction of Fanconi anemia in human hematopoietic cells with a recombinant adeno-associated virus vector,” J. Clin. Invest, 94:1440-1448, 1994.

Wang, Cardiff, Zukerberg, Lees, Arnold, Schmidt, “Mammary hyperplasia and carcinoma in MMTV-cyclin D1 transgenic mice,” Nature, 369:669-671, 1994.

Wei, Wei, Samulski, and Barranger, “Expression of the human glucocerebrosidase and arylsulfatase A genes in murine and patient primary fibroblasts transduced by an adeno-associated virus vector,” Gene Therapy, 1:261-268, 1994.

White, Adv. Pharmacol, 40:339-67, 1997

Wong, G. T., Gavin, B. J. & McMahon, A. P. (1994) Mol. Cell. Biol. 14, 6278-6286.

Yang, Chen, Trempe, “Characterization of cell lines that inducibly express the adeno-associated virus Rep proteins,” J. Virol, 68:4847-4856, 1994.

Yoder, Kang, Zhou, Luo, and Srivastava, “In vivo gene transfer in murine hematopoietic reconstituting stem cells mediated by the adeno-associated virus 2-based vectors,” Blood, 82 (Supp.): 1:347A, 1994.

Zhou, Cooper, Kang, Ruggieri, Heimfeld, Srivastava, and Broxmeyer, “Adeno-associated virus 2-mediated high efficiency gene transfer into immature and mature subsets of hematopoietic progenitor cells in human umbilical cord blood,” J.Exp.Med., 179:1867-1875, 1994.

What is claimed is:

1. A method for determining an existence of or characterizing cancer in a subject comprising:
(a) obtaining a sample from said subject; and
(b) analyzing said sample for an increase or decrease in β-catenin activation.

2. The method of claim 1, further defined as a method of determining a prognosis of a subject with cancer.

3. The method of claim 1, further defined as a method of identifying a subject at risk of development, recurrence, or metastasis of cancer.

4. The method of claim 1, further defined as a method of determining a survival rate of a patient with cancer.

5. The method of claim 1, wherein said cancer is breast cancer.

6. The method of claim 1, wherein said cancer is ovarian cancer.

7. The method of claim 1, wherein said cancer is colon cancer.

8. The method of claim 1, wherein the sample comprises a cell.

9. The method of claim 1, wherein said sample comprises a fine needle aspiration, a core needle biopsy, a surgical biopsy, a vacuum-assisted biopsy, a biopsy using an advanced breast biopsy instrument, a surgical specimen, a paraffin embedded tissue or a frozen tissue imprint.

10. The method of claim 8, wherein analyzing the sample for an increase or decrease in β-catenin activation comprises determining subcellular localization of β-catenin at a cytoplasm, nucleus or plasma membrane of said cell.

11. The method of claim 10, wherein a decrease in β-catenin activation is determined by β-catenin localization at a plasma membrane.

12. The method of claim 11, wherein said decreased β-catenin activation indicates an increase in patient survival rate.

13. The method of claim 10, wherein an increase in β-catenin activation is determined by β-catenin accumulation at a cytoplasm or nucleus.

14. The method of claim 13, wherein said increased β-catenin activation indicates a decrease in patient survival rate.

15. The method of claim 8, wherein analyzing the sample comprises:
(a) transfecting the cell with a cyclin D1 reporter;
(b) transfecting the cell with a β-catenin-encoding nucleic acid; and
(c) analyzing the transfected cell.

16. The method of claim 15, wherein said cyclin D1 reporter comprises a Tcf4 wild-type reporter.

17. The method of claim 15, wherein said cyclin D1 reporter comprises a Tcf4 mutant reporter.

18. The method of claim 15, further comprising using antibodies to one or more of β-catenin, cyclin D1 and α-actin.

19. The method of claim 15, wherein said β-catenin in the β-catenin-encoding nucleic acid is a phosphorylation mutant.

20. The method of claim 15, wherein said β-catenin in the β-catenin-encoding nucleic acid is myc tagged.

21. The method of claim 15, further comprising transfecting said sample with a control vector.

22. The method of claim 21, wherein said control vector is pcDNA3.

23. The method of claim 15, further comprising transfecting said sample with a negative β-catenin/Tcf regulator.

24. The method of claim 1, wherein analyzing the sample comprises immuno-histochemical staining.

25. The method of claim 1, wherein analyzing the sample comprises immunoblot analysis.

26. The method of claim 14, further comprising instituting a more aggressive treatment protocol in said patient with an increase in β-catenin activation.

27. The method of claim 1, further comprising blocking the β-catenin activation in said patient with an increase in β-catenin activation.

28. The method of claim 1, further comprising blocking Tcf4 function in said patient with an increase in β-catenin activation.

29. A method for identifying a patient at risk for the development, recurrence, or metastasis of cancer and/or determining the prognosis of a patient with cancer comprising:
(a) obtaining a sample comprising a cell from a patient;
(b) transfecting the cell with cyclin D1 reporter;
(c) transfecting the cell with β-catenin-encoding nucleic acid; and
(d) analyzing said transfected cell.

30. The method of claim 29, wherein said cancer is breast cancer.

31. The method of claim 29, wherein analyzing comprises determining whether the cell has β-catenin subcellular localization at a cytoplasm, nucleus or plasma membrane.

32. The method of claim 31, wherein a decrease in activation is determined by β-catenin localization at a plasma membrane.

33. The method of claim 32, wherein said decreased β-catenin activation indicates an increase in patient survival rate.

34. The method of claim 31, wherein an increase in β-catenin activation is determined by β-catenin accumulation at a cytoplasm or nucleus.

35. The method of claim 34, wherein said increased β-catenin activation indicates a decrease in patient survival rate.

36. The method of claim 31, further comprising treating said patient after identification of development, recurrence, or metastasis of cancer.

37. A method for cancer therapy in a patient comprising modulating β-catenin activation in a patient.

38. The method of claim 37, wherein said cancer is breast, ovarian, or colon cancer.

39. The method of claim 37, comprising blocking β-catenin activation in said patient.

40. The method of claim 37, comprising blocking the Tcf4 function in said patient.

41. The method of claim 37, comprising contacting the patient with an antisense of β-catenin, a dominant-negative mutant of β-catenin, a dominant-negative Tcf4, an antisense of Tcf4, or a combination thereof.

42. The method of claim 37, comprising contacting the patient with a small molecule that modulates β-catenin activation.
